(19) United States
(12) Patent Application Publication
(10) Pub. No.: US 2013/0338368 A1
(43) Pub. Date: Dec. 19, 2013

(54) METHOD FOR ANALYSIS OF PROTEIN AND ANALYTICAL REAGENT

(71) Applicants: Kanto Kagaku Kabushiki Kaisha, Tokyo (JP); National Institute of Advanced Industrial Science and Technology, Tokyo (JP)

(72) Inventors: Yoshio Suzuki, Tsukuba-shi (JP); Nobuyuki Takagi, Tokyo (JP); Tomoyuki Chimuro, Isehara-shi (JP); Takuma Sano, Isehara-shi (JP)

(21) Appl. No.: 13/916,773

(22) Filed: Jun. 13, 2013

(30) Foreign Application Priority Data
Jun. 13, 2012 (JP) 2012-134262

(51) Int. Cl. C07D 405/14; G01N 21/64; C07D 401/14

(52) U.S. Cl. CPC C07D 405/14; C07D 401/14; G01N 21/6486
USPC 546/256; 546/264; 546/265

(57) ABSTRACT

An object of the present invention is to provide a reagent for protein analysis that has still greater 1) rapidity and 2) high operability, that can be detected with 3) high sensitivity, and that can be detected using 4) a gel imager that carries out excitation with visible light or a detecting device having a highly versatile UV light source (UV transilluminator), and a method for protein analysis.

The present invention relates to a composition for analysis of a protein, the composition containing a compound represented by Formula I

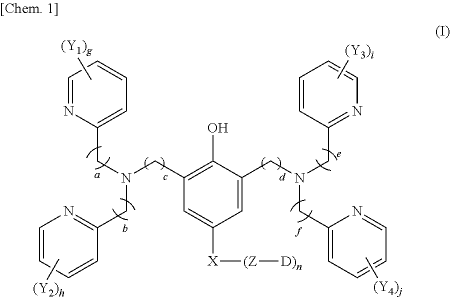

or a salt thereof; when the composition is applied to a support containing a separated protein, staining and fixation of the protein being carried out thereby, and no destaining of the support after application being required.

### TECHNICAL FIELD

The present invention relates to a composition, containing a specific fluorescent compound, for analysis of a protein, a method for analysis of a protein using the composition, a fluorescent compound contained in the composition, etc.

### BACKGROUND ART

As a method for analyzing a protein, electrophoresis is widely carried out. The main purpose thereof is to measure the molecular weight of a protein or to prepare a sample for protein identification. A protein separated by electrophoresis normally needs to be reacted with a reagent that interacts with the protein, before electrophoresis or after electrophoresis, so as to make it possible for the protein to be detected by eye or by a dedicated detector. Many types of methods and reagents for detecting a protein with high sensitivity have been developed so far and put into practice. Examples thereof include silver staining and SYPRO Ruby staining.

Silver staining is a highly sensitive method having a detection sensitivity level of 1 to 10 ng but has the defects that it requires a longtime (120 min. or longer), it is not quantitative, and it requires silver ion effluent to be treated. A method employing a fluorescent dye such as SYPRO Ruby (Life Technologies Corporation) might exhibit a detection sensitivity higher than that of silver staining, but in addition to it requiring a longtime (240 min. or longer), when it is applied to SDS-PAGE, there is a large effect from residual SDS in a gel, and a target protein might not be detected. Furthermore, since the detection sensitivity is greatly affected by excess fluorescent dye remaining in the gel, it is usually necessary to carry out procedures of fixing a protein in the gel and washing a sufficient number of times before and after reacting the fluorescent dye, and it has been pointed out that the rapidity is impaired and the burden on an operator is high. Therefore, as described in Patent Document 1 and Non-Patent Document 1, ‘Rapid FluoroStain KANTO’, which eliminates the above defects, has been developed.

Following this, Oriole staining (Bio-Rad Laboratories, Inc.) has been developed. Oriole staining does not require a fixing operation after electrophoresis or a destaining operation after staining and is one-shot staining where staining is possible simply by immersing a gel in a staining liquid. However, since the time required is as long as 90 min. or longer, and the gel shrinks after staining, it might be difficult to discriminate between adjacent protein spots. Furthermore, it cannot be used for Native-PAGE, and fluorescence detection by excitation with visible light is impossible, thus limiting the application thereof.

On the other hand, Rapid FluoroStain KANTO has a shorter operating process time (about 60 min.) compared with Oriole staining, and shrinkage of a gel after staining is not observed, but one-shot staining cannot be carried out.

Patent Document 2 describes a metal complex used in a staining method for a poly(amino acid) where the necessity for destaining and fixation is eliminated. However, a staining method using this complex requires about 90 min. in order to obtain the maximum staining intensity, the time required thus being long. Furthermore, since this staining method employs methanol, which is designated as a non-medical deleterious substance, it cannot be said to have excellent safety.

As hereinbefore described, a staining method that can carry out detection with high sensitivity and has good operability is still desired.

### PRIOR ART DOCUMENTS

### Patent Documents

[Patent Document 1] JP, A, 2010-014673
[Patent Document 2] JP, A, 2011-505342

### Non-Patent Documents

[Non-Patent Document 1] Electrophoresis 2011, 32, 1403-1413

### SUMMARY OF THE INVENTION

### Problems to be Solved by the Invention

It is an object of the present invention to provide a reagent for protein analysis that has still greater 1) rapidity and 2) high operability, that can be detected with 3) high sensitivity, and that can be detected using 4) a gel imager that carries out excitation with visible light or a detecting device having a highly versatile UV light source (UV transilluminator), and a method for protein analysis.

### Means for Solving the Problems

While carrying out an intensive investigation in order to solve the above-mentioned problems, the present inventors have found that by using a composition containing the fluorescent compound of the present invention, a protein can be detected easily and with high sensitivity by means of a gel imager that carries out excitation with UV and visible light, and as a result of further research the present invention has been accomplished.

That is, the present invention relates to the following.

(1) A composition for analysis of a protein, containing one or more types of compound represented by Formula I and/or salt thereof

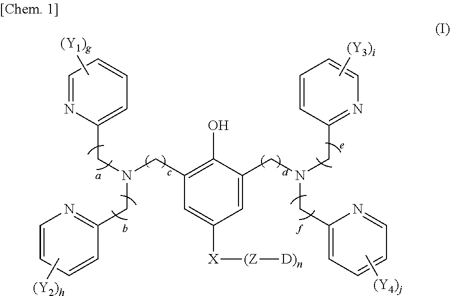

wherein in the Formula a to f are mutually independently 1 to 5,
Y₁ is a straight-chain or branched alkyl group having 1 to 10 C atoms, a carboxyl group, a hydroxy group, an amino group, a thiol group, or a halogen, and when a plurality of Y₁s are present they may be mutually independently identical or different,
Y₂ to Y₄ mutually independently denote the same as Y₁,
g, h, i, and j are mutually independently 0 to 4,
X is a straight-chain hydrocarbon group having 1 to 10 C atoms in which one or more hydrogen atoms are mutually independently and optionally replaced by —COOH, —OH, —NH₂, R₁, —OR₁, —COR₁, —COOR₁, —CONHR₁, —SH, or a halogen, R₁ being an alkyl group having 1 to 5 C atoms, and at least one hydrogen atom bonded to a terminal carbon atom of the hydrocarbon group being replaced by —Z-D,
n is 1 to 3,
Z is a single bond or an alkylene group, when a plurality of Zs are present they are independent from each other, one or more —CH₂— moieties being mutually independently and optionally replaced by —O—, —S—, —NH—, —NH—SO₂—, —SO₂—NH—, —CS—NH—, —NH—CS—, —CO—NH—, —NH—CO—, —CO—, —COO—, —OCO—, —OCO—O—, —S—CO—, —CO—S—, —SO₂—, —CH═CH—, or —C≡C—, and
D is a chromophore, when a plurality of Ds are present they are independent from each other,
when the composition is applied to a support containing a separated protein, staining and fixation of the protein being carried out thereby, and no destaining of the support after application being required.
(2) The composition according to (1), wherein D is selected from the group consisting of

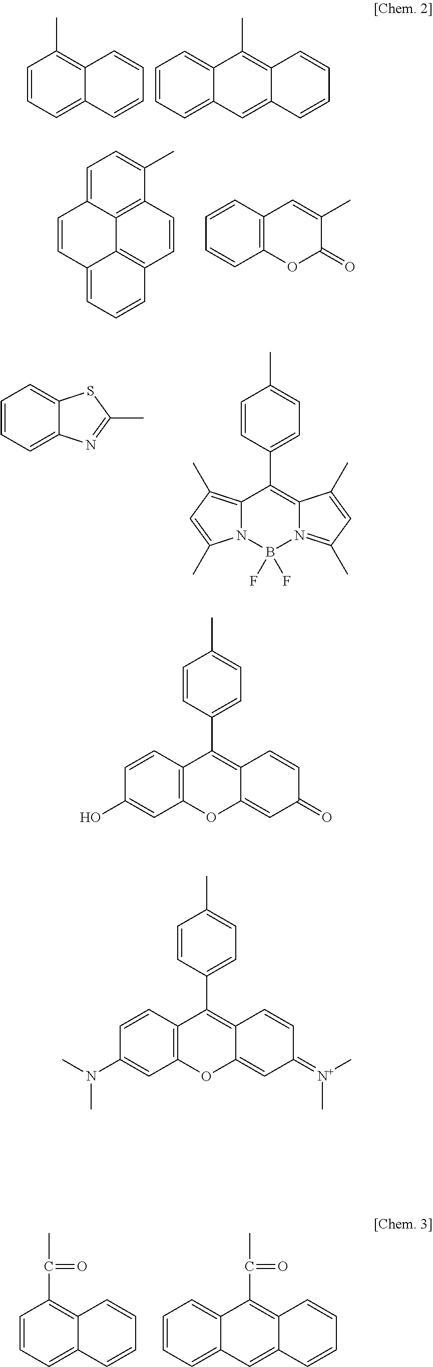

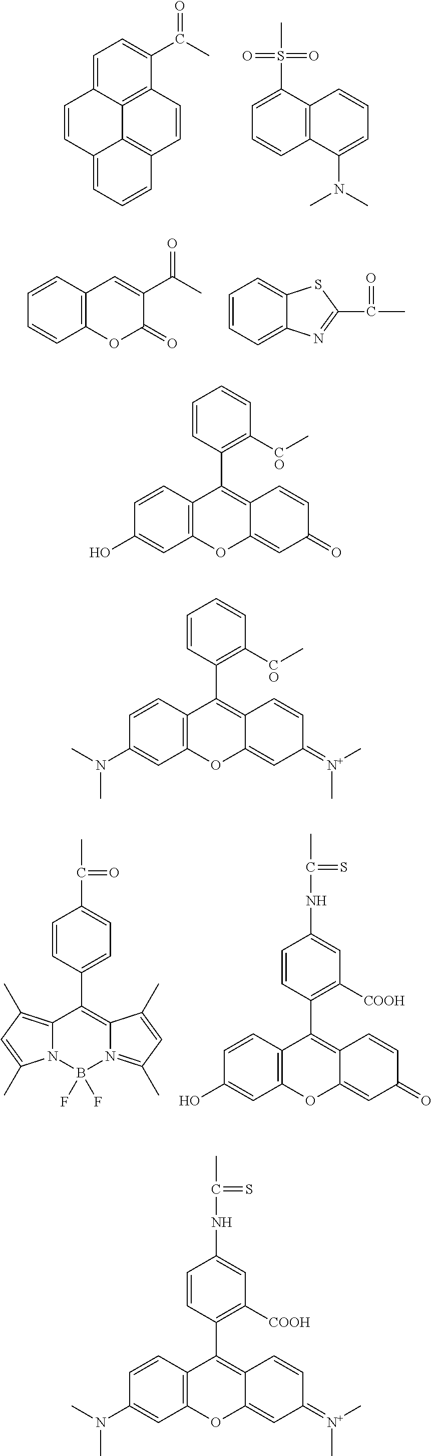

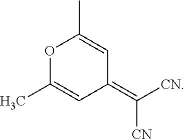

(3) The composition according to (1) or (2), wherein —X—(Z-D)ₙ is selected from the group consisting of those below

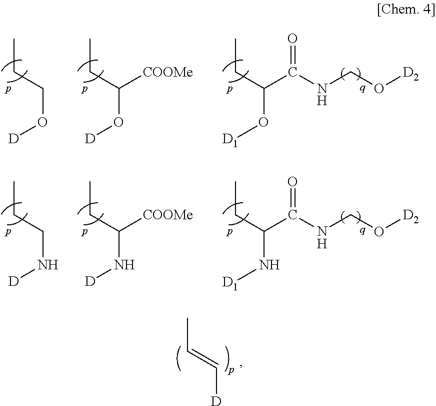

D is selected from the group consisting of

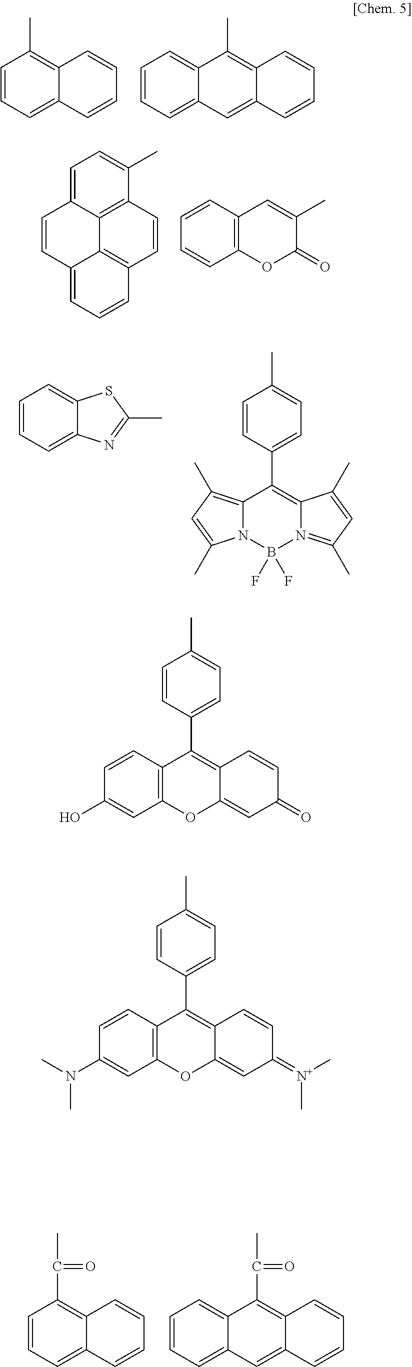

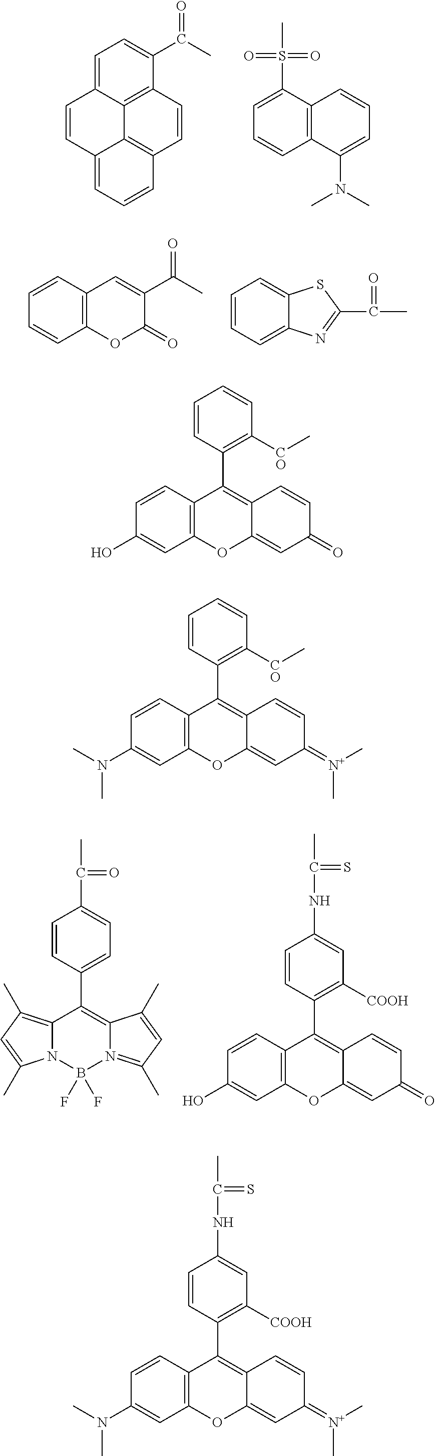

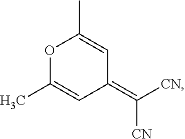

D₁ and D₂ mutually independently denote the same as D, and
p and q are mutually independently a number of 1 to 10.
(4) The composition according to any one of (1) to (3), wherein it comprises at least one type of compound represented by Formulae Ia to Ic and/or salt thereof

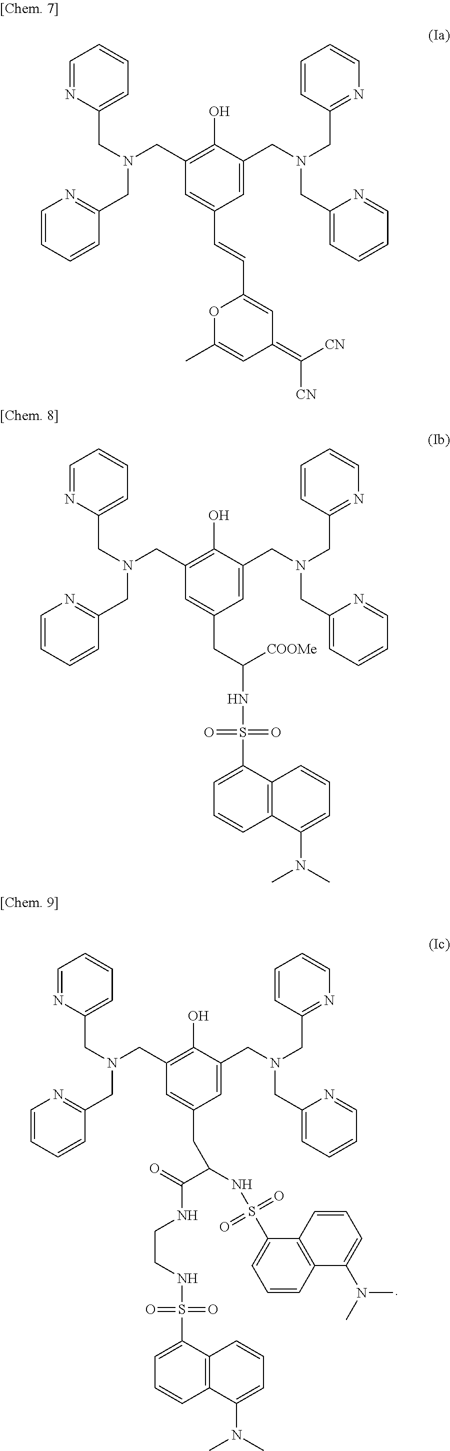 [SMILES: CC1=CC(=C(C#N)C#N)C=C(/C=C/c2cc(CN(Cc3ccccn3)Cc3ccccn3)c(O)c(CN(Cc3ccccn3)Cc3ccccn3)c2)O1 ; COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12 ; CN(C)c1cccc2c(S(=O)(=O)NCCNC(=O)C(Cc3cc(CN(Cc4ccccn4)Cc4ccccn4)c(O)c(CN(Cc4ccccn4)Cc4ccccn4)c3)NS(=O)(=O)c3cccc4c(N(C)C)cccc34)cccc12]

(5) A method for analysis of a protein, the method including a step of separating a protein on a support by means of electrophoresis and a step of carrying out staining and fixation of the protein by immersing the support in an aqueous solution containing the composition according to any one of (1) to (3), but not comprising a step of destaining the support after immersion.
(6) The method according to (5), wherein it further includes a step of subjecting the support after immersion to fluorescence image analysis by illuminating with visible light or UV.
(7) A compound represented by

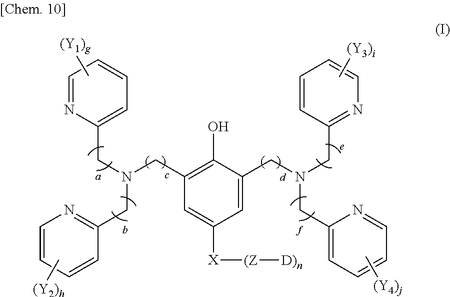

or a salt thereof,
wherein in the Formula
—X—(Z-D)ₙ is selected from the group consisting of those below

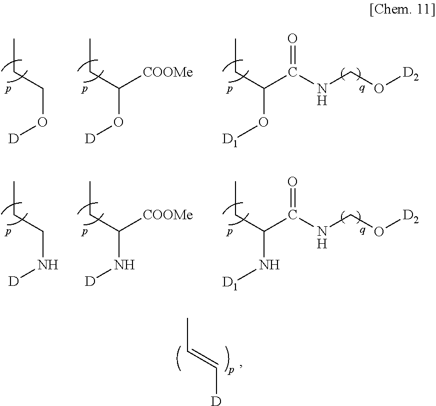

D is selected from the group consisting of

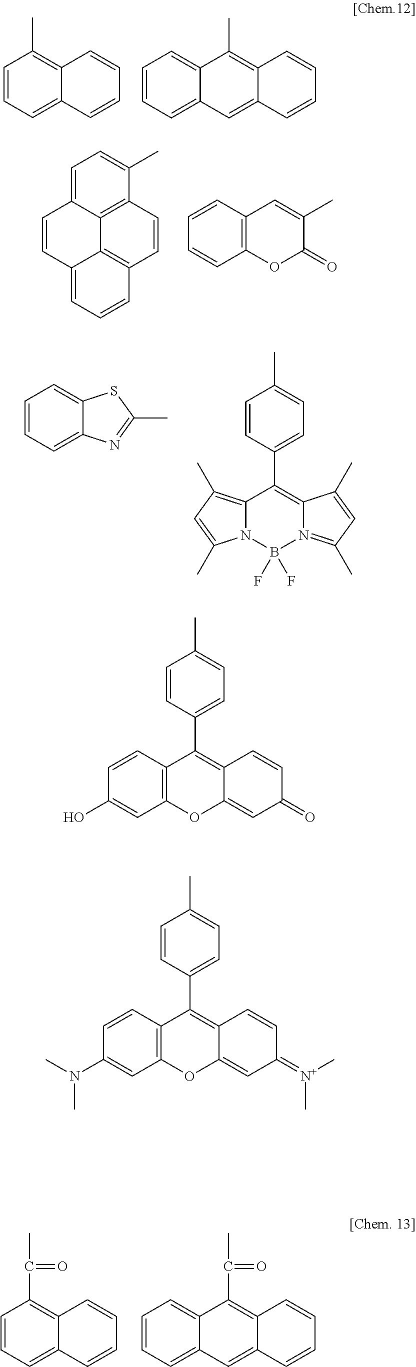

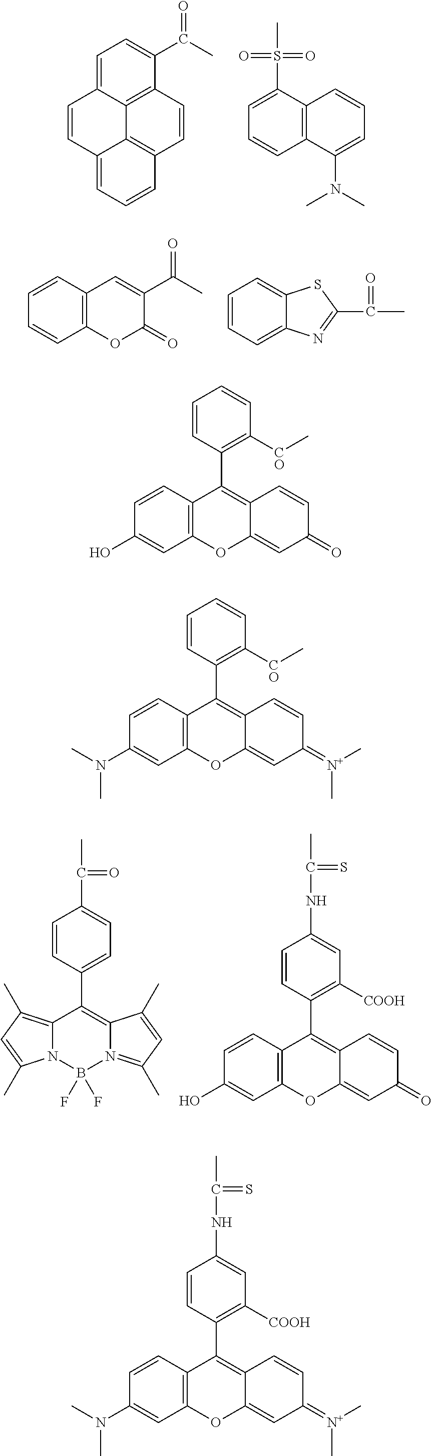

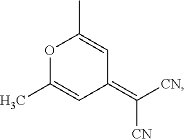

D₁ and D₂ mutually independently denote the same as D, and
p and q mutually independently denote a number of 1 to 10.
(8) The compound or salt thereof according to 7, wherein the compound is represented by Formula Ia

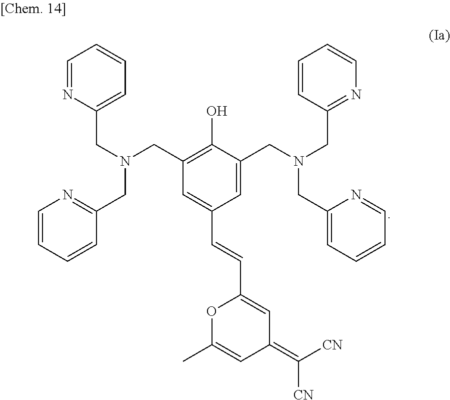 [SMILES: CC1=CC(=C(C#N)C#N)C=C(/C=C/c2cc(CN(Cc3ccccn3)Cc3ccccn3)c(O)c(CN(Cc3ccccn3)Cc3ccccn3)c2)O1]

(9) The compound or salt thereof according to 7, wherein the compound is represented by Formula Ib

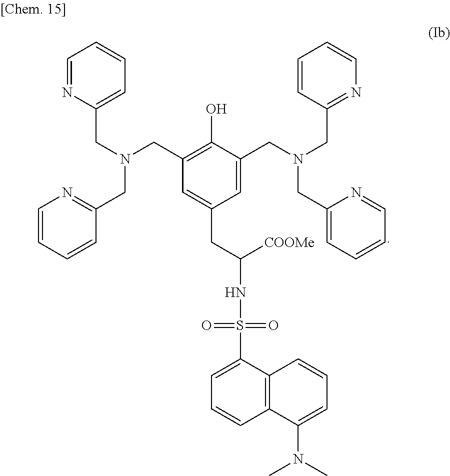 [SMILES: COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12]

(10) The compound or salt thereof according to 7, wherein the compound is represented by Formula Ic

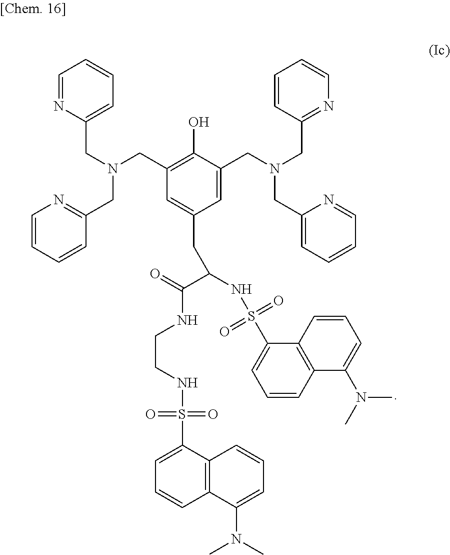 [SMILES: CN(C)c1cccc2c(S(=O)(=O)NCCNC(=O)C(Cc3cc(CN(Cc4ccccn4)Cc4ccccn4)c(O)c(CN(Cc4ccccn4)Cc4ccccn4)c3)NS(=O)(=O)c3cccc4c(N(C)C)cccc34)cccc12]

### Effects of the Invention

In accordance with use of the composition of the present invention, it becomes possible to detect a protein in an electrophoresis support simply, rapidly and with high sensitivity. Therefore, compared with prior techniques the present invention enables analysis to be carried out in a short period of time with high precision without requiring complicated operations even when a large number of samples are analyzed, as in proteome analysis. Furthermore, a support (gel, etc.) that has been subjected to Native-PAGE (a method in which SDS (sodium dodecyl sulfate), which is a surfactant, is not used and a protein is subjected to gel electrophoresis without being modified) can easily be stained, and the range of applications is wide.

### BRIEF DESCRIPTION OF DRAWINGS

FIG. 1 is a diagram of fluorescence imaging after electrophoresis in Example 5 (fluorescent dye concentration: 16 μg/mL Fluorescent Substance 1; staining liquid: phosphoric acid buffer/purified water/methanol; excitation wavelength 525 nm; cut-off filter R-60).

FIG. 2 is a diagram of fluorescence imaging after electrophoresis in Example 6 (fluorescent dye concentration: 16 μg/mL Fluorescent Substance 2; staining liquid: glycine buffer/purified water/ethanol; excitation wavelength 365 nm; cut-off filter SCF515).

FIG. 3 is a diagram of fluorescence imaging after electrophoresis in Example 7 (fluorescent dye concentration: 16 μg/mL Fluorescent Substance 3; staining liquid: glycine buffer/purified water/ethanol; excitation wavelength 365 nm; cut-off filter SCF515).

FIG. 4 is a diagram of fluorescence imaging after Native-PAGE in Example 8 (fluorescent dye concentration: 16 μg/mL Fluorescent Substance 2; staining liquid: glycine buffer/purified water/ethanol; excitation wavelength 312 nm; cut-off filter SCF515).

FIG. 5 is a diagram of fluorescence imaging after Native-PAGE in Example 8 (fluorescent dye concentration: 16 μg/mL Fluorescent Substance 2; staining liquid: glycine buffer/purified water/ethanol; excitation wavelength 365 nm; cut-off filter SCF515).

### MODES FOR CARRYING OUT THE INVENTION

One aspect of the present invention relates to the above composition, which is a reagent for analysis of a protein, the composition containing a compound represented by Formula (I) above or a salt thereof, being applied to a support containing a separated protein to thus carry out staining and fixation of the protein, and not requiring destaining of the support after being applied.

In the present invention, ‘analysis of a protein’ includes detecting a protein qualitatively and comprehensively, that is, confirming the presence or absence of a protein, measuring the molecular weight of a protein, determining the distribution of a protein, and carrying out a quantitative measurement.

The composition for protein analysis of the present invention contains one or more types of compound represented by Formula (I) and/or salt thereof.

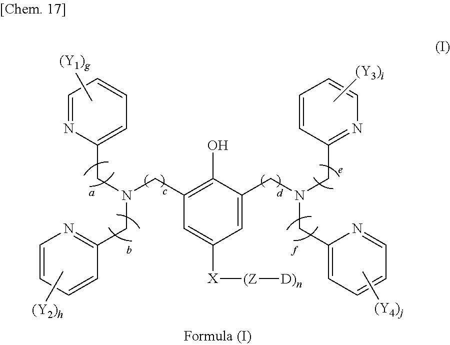

In the Formula, a to f are mutually independently 1 to 5, and preferably 1 to 3,
Y₁ is a straight-chain or branched alkyl group having 1 to 10 C atoms, and preferably 1 to 3 C atoms, a carboxyl group, a hydroxy group, an amino group, a thiol group, or a halogen, and when a plurality of Y₁s are present, they may be mutually independently identical or different,
Y₂ to Y₄ mutually independently denote the same as Y₁, and g, h, i, and j are mutually independently 0 to 4, and preferably 0 or 1.

X is a straight-chain hydrocarbon group typically having 1 to 10, and preferably 1 to 3 C atoms, one or more hydrogen atoms may be mutually independently replaced by typically —COOH, —OH, —NH₂, —R₁, —OR₁, —COR₁, —COOR₁, —CONHR₁, —SH, or a halogen, and at least one hydrogen atom bonded to a terminal carbon atom of the hydrocarbon group may be replaced by —Z-D.

R₁ is an alkyl group having 1 to 5 C atoms, and is preferably a methyl group (Me) or an ethyl group (Et).

The hydrocarbon is an alkyl group, an alkenyl group, or an alkynyl group, wherein they typically have 1 to 10, and preferably 1 to 3 C atoms. Specific examples of X include, but are not limited to, an alkyl group having 1 to 3 C atoms, an alkenyl group having 1 to 3 C atoms, an alkynyl group having 1 to 3 C atoms, and an alkyl group having 1 or 2 C atoms in which one or more hydrogen atoms are replaced by —COOR₁, and preferably —COOMe and/or —COOEt.

In Formula (I), n is 1 to 3.

In Formula (I), Z is typically a single bond or an alkylene group, when a plurality of Zs are present they are independent from each other, said alkylene group preferably having 1 to 10, and particularly preferably 1 to 3C atoms, and one or more —CH₂— moieties may be mutually independently replaced by —O—, —S—, —NH—, —NH—SO₂—, —SO₂—NH—, —CS—NH—, —NH—CS—, —CO—NH—, —NH—CO—, —CO—, —COO—, —OCO—, —OCO—O—, —S—CO—, —CO—S—, —SO₂—, —CH═CH—, or —C≡C—. When a plurality of Zs are present, they may be identical to or different from each other. From the viewpoint of bond stability under acidic or basic conditions, Z is preferably an alkylene group in which one or more —CH₂— moieties are replaced by —CO—, —NH—, —CO—NH—, —NH—CO—, —NH—SO₂—, and/or —SO₂—NH—.

When n=1, examples of —X—(Z-D) include, but are not limited to

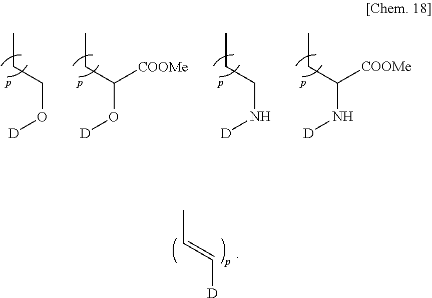

When n=2, examples of —X—(Z-D)₂ include, but are not limited to

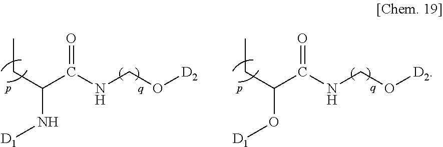

In Formula (I), D is a chromophore, and when a plurality of Ds are present they are independent from each other. When a plurality of Ds are present, they may be identical to or different from each other. The chromophore of the present invention is typically a functional group that absorbs electromagnetic waves in the UV-VIS (ultraviolet-visible) region (preferably 200 to 830 nm). In the present invention, ‘UV’ means UV light or a UV beam having a wavelength of 200 to 380 nm, and ‘visible light’ means visible light or a visible light beam having a wavelength of 380 to 830 nm.

Specific examples of the chromophore include, but are not limited to, a fluorescein group and a derivative thereof, a dansyl group and a derivative thereof, a coumarin group and a derivative thereof, a pyrene group and a derivative thereof, an anthracene group and a derivative thereof, a rhodamine group and a derivative thereof, a benzothiazole group and a derivative thereof, and a boron-dipyrromethene group and a derivative thereof. The derivative means one formed by further substituting the respective functional group with a substituent, and examples of such a substituent include, but are not limited to, an alkyl group having 1 to 5 C atoms, a carbonyl group, a carboxyl group, an —OH group, an isothiocyanate group, a halogen group, an amino group, a thiol group, and an ether group.

Specific examples of D include, but are not limited to, the functional groups below.

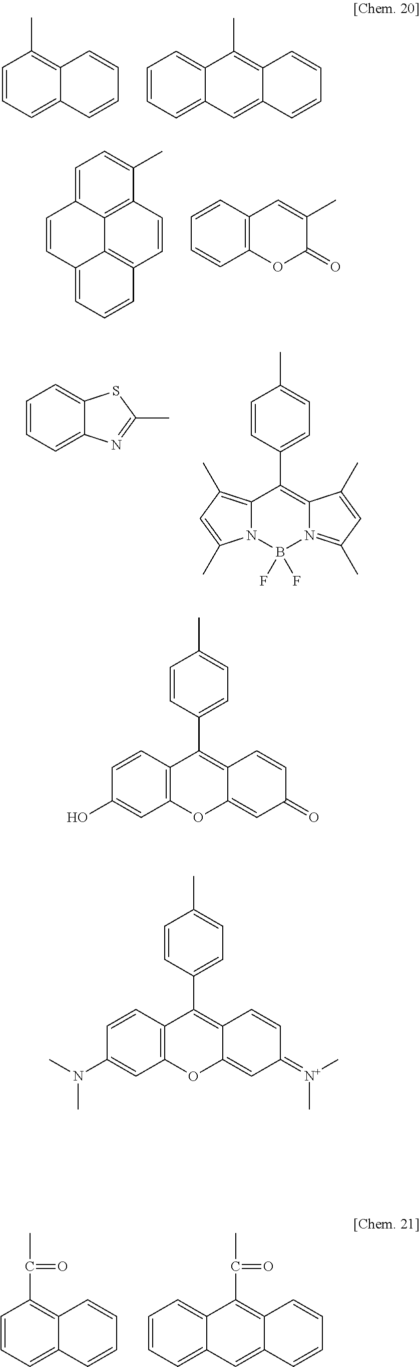

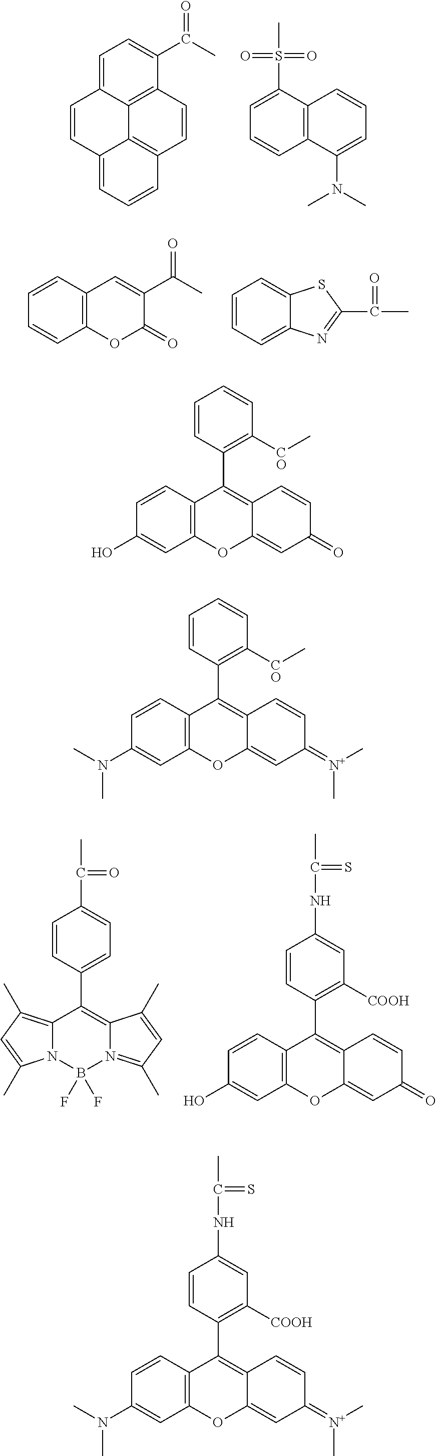

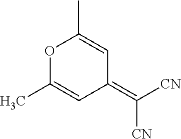

Namely, D may be 1-naphthyl, 9-anthracenyl, 1-pyrenyl, 3-coumarinyl, 2-benzothiazolyl, 1-borondipyromethanyl, 4-fluroescenyl, N,N,N′,N′-tetramethyl-4-rhodaminyl, Naphthalene-1-carbonyl, Anthracene-9-carbonyl, Pyrene-1-carbonyl, Dansyl, Coumarin-3-carbonyl, Benzothiazol-2-carbonyl, Fluorescein-2-carbonyl, N,N,N′,N′-tetramethyl-rhodamine-2-carbonyl, Borondipyromethane-1-carbonyl, Fluorescein-2-carboxylic acid-4-thioureanyl, N,N,N′,N′-tetramethyl-rhodamine-2-carboxylic acid-4-thioureanyl, 4-(dicyanomethylene)-2-methyl-4H-pyranyl and so on.

In one preferred embodiment of the composition of the present invention, the composition of the present invention contains one or more types of compound represented by the following Formula and/or salt thereof

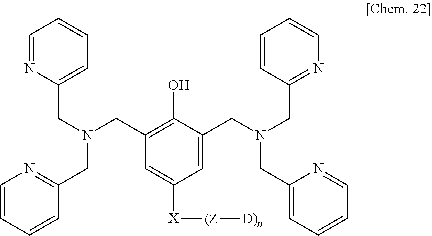

wherein in the Formula, —X—(Z-D)ₙ is selected from the group consisting of those below

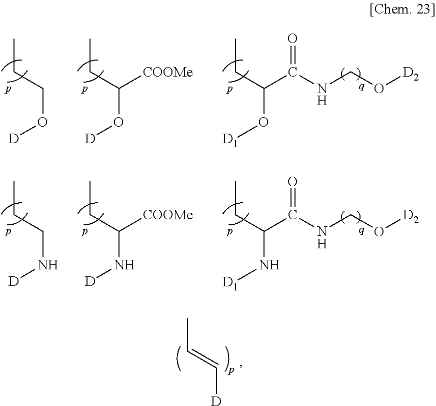

D is selected from the group consisting of

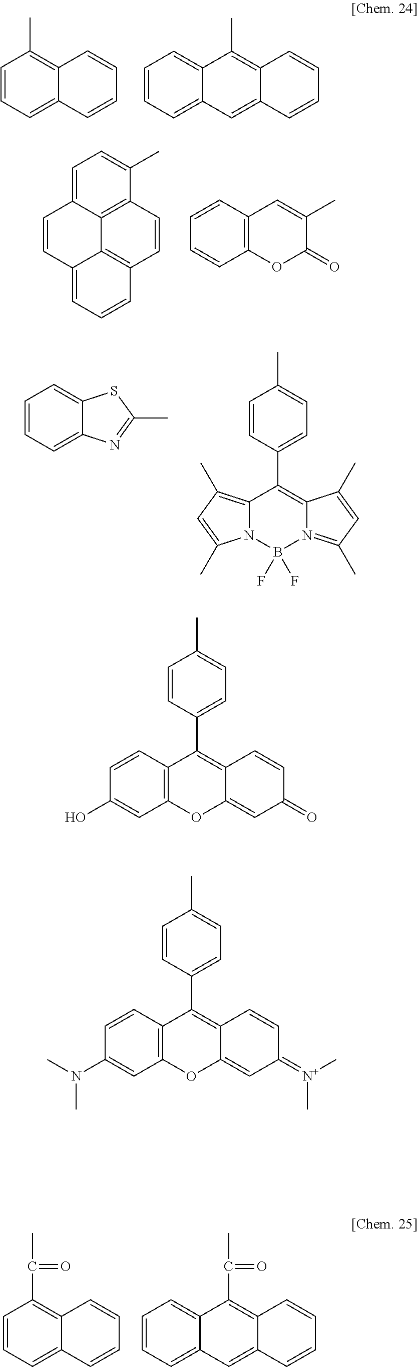

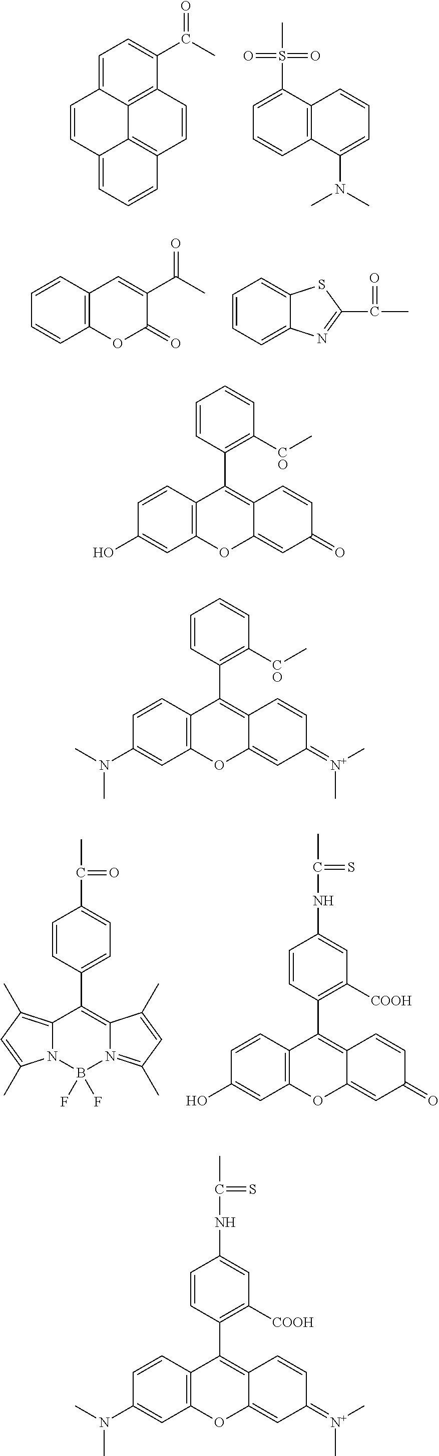

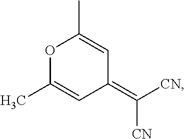

D₁ and D₂ mutually independently denote the same as D, and p and q are mutually independently 1 to 10. Preferably, p and q are mutually independently 1 to 3.

One preferred embodiment of the composition of the present invention contains one or more types of compound represented by Formula (I) above and/or salt thereof, wherein in the Formula —X—(Z-D)ₙ is selected from the group consisting of

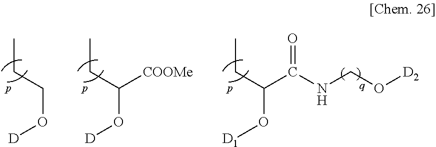

and
D is selected from the group consisting of

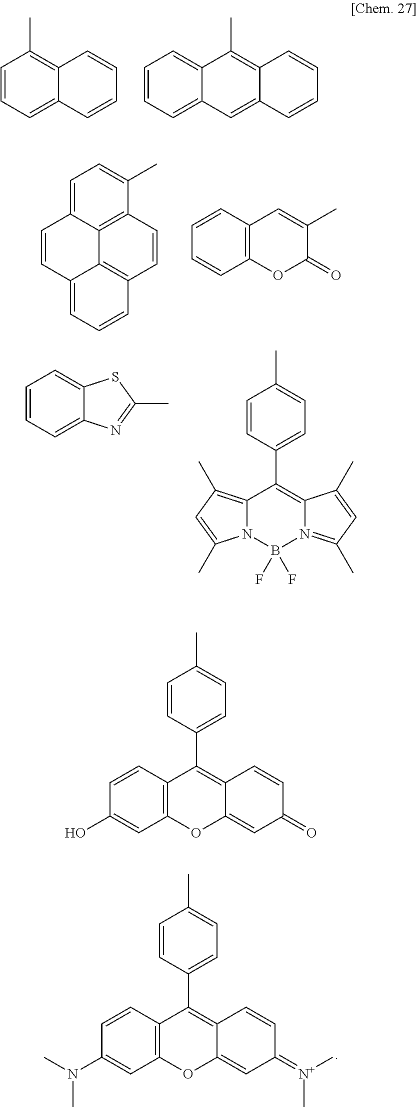

One preferred embodiment of the composition of the present invention contains one or more types of compound represented by Formula (I) above and/or salt thereof, wherein in the Formula —X—(Z-D)ₙ is selected from the group consisting of

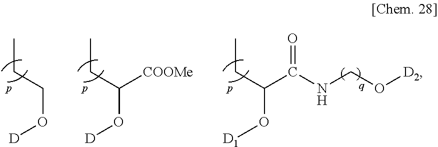

and
D is selected from the group consisting of

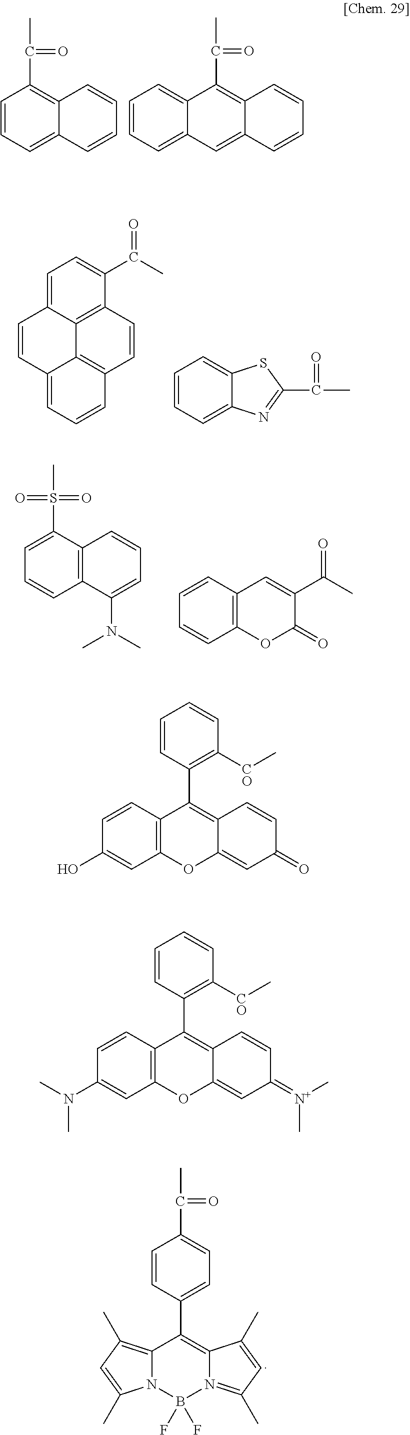

One preferred embodiment of the composition of the present invention contains one or more types of compound represented by Formula (I) above and/or salt thereof, wherein in the Formula —X—(Z-D)ₙ is selected from the group consisting of

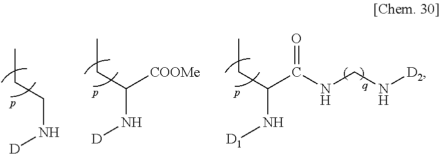

D is selected from the group consisting of

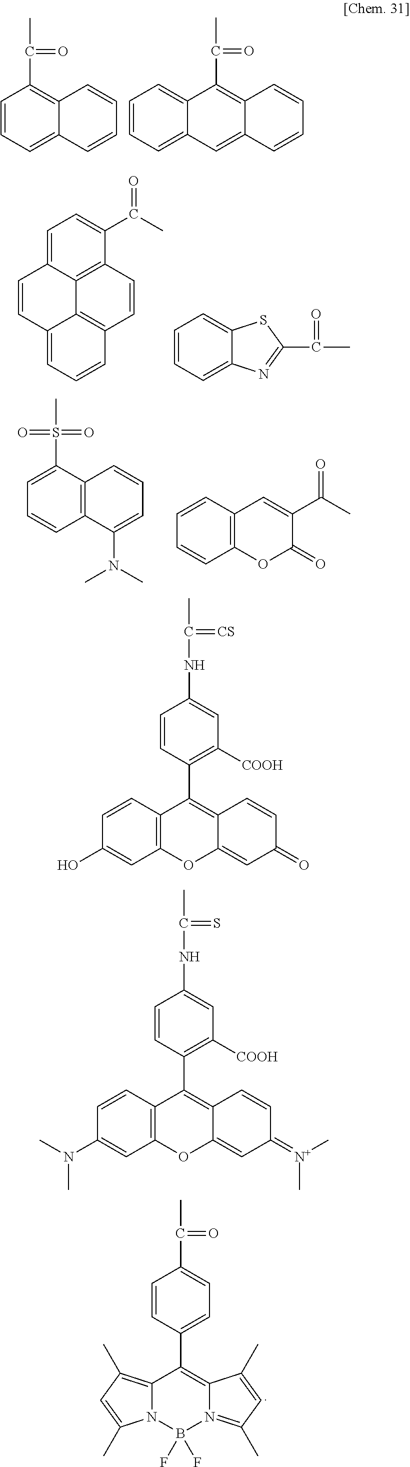

One preferred embodiment of the composition of the present invention contains one or more types of compound represented by Formula (I) above and/or salt thereof, wherein in the Formula —X—(Z-D)ₙ is

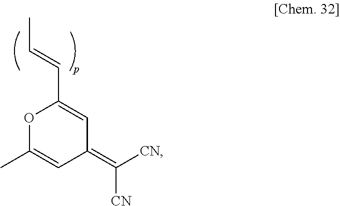

and
p is 1 to 5.

In a preferred embodiment of the composition of the present invention, the composition of the present invention contains one or more types of compound represented by at least one type of Formulae Ia to Ic and/or salt thereof

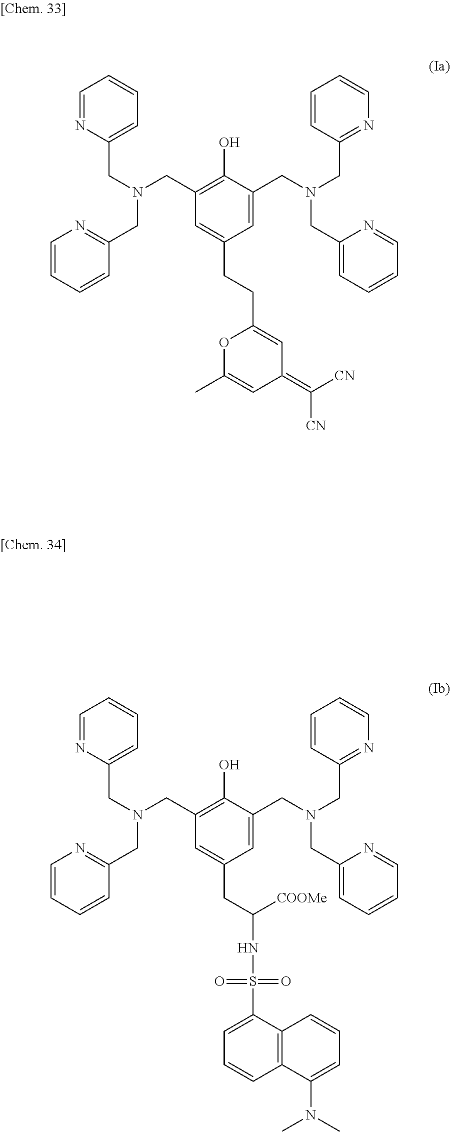 [SMILES: CC1=CC(=C(C#N)C#N)C=C(CCc2cc(CN(Cc3ccccn3)Cc3ccccn3)c(O)c(CN(Cc3ccccn3)Cc3ccccn3)c2)O1 ; COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12]

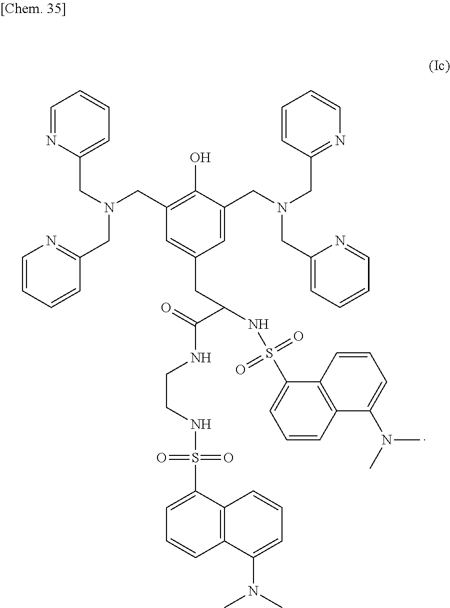 [SMILES: CN(C)c1cccc2c(S(=O)(=O)NCCNC(=O)C(Cc3cc(CN(Cc4ccccn4)Cc4ccccn4)c(O)c(CN(Cc4ccccn4)Cc4ccccn4)c3)NS(=O)(=O)c3cccc4c(N(C)C)cccc34)cccc12]

The composition of the present invention contains a compound represented by Formula (I) and/or a salt thereof and may contain one or more types. Examples of the salt include, but are not limited to, a complex in which a transition metal ion (zinc, cobalt, nickel, copper, cadmium, mercury, iron, manganese) coordinates to a pyridine ring and a salt in which H of a phenolic hydroxy group is replaced by Na or K.

The compound represented by Formula (I) of the present invention is a fluorescent compound. In the present invention, ‘fluorescence’ means a property of typically generating, when illuminated with light (excitation light) having a specific wavelength, light (fluorescence) having a maximum wavelength that is longer than that of the light with which it is illuminated. The maximum wavelength of the fluorescence of the compound (fluorescent compound) in accordance with the present invention is not particularly limited, and is for example 450 to 600 nm. Measurement of fluorescence may be carried out using various types of instrument. For example, measurement may be carried out by means of a commercial fluorescence image analyzer (available from Atto Corporation, TECAN, GE Healthcare, etc.).

The composition of the present invention may simply be applied to a support containing a separated protein, thus carrying out staining and fixation of the protein, and destaining of the support after application is not necessary. Furthermore, a step of fixing a protein to a gel, that is, a step of fixing for a support before and after staining or a step of washing the support is not required. For example, after a sample containing a protein is subjected to electrophoresis to thus carry out separation, the support containing the separated protein, for example, an electrophoresis support such as a gel, may simply be immersed in the composition of the present invention, and it is thus possible to carry out staining and fixation of the protein. A destaining step for removing excess staining composition remaining on a support, which is an essential step for the conventional SYPRO Ruby method, etc., is not necessary. Therefore, the staining step is completed simply by immersing the support, and quantitative or qualitative analysis can be carried out as it is by naked eye or by an imaging analysis employing excitation by UV or visible light. In this way, completion of the staining step in one step without a fixation step, washing step, or destaining step being required is called ‘one-shot staining’ in the present specification.

Although the reason why the composition of the present invention can make one-shot staining possible is not necessarily clear, it is surmised that the compound represented by Formula (I) binds to a protein to thus increase the intensity of fluorescence. It is thought that due to —X—(Z-D)ₙ and a pyridyl group in Formula (I) strongly binding specifically to a protein, the protein is fixed. It is surmised that the fluorescence intensity of the compound itself represented by Formula (I) remaining on the support is very small compared with that of one bound to a protein, a low background can be achieved, and detection of a protein with high sensitivity becomes possible at an excellent S/N ratio. Although the reason why very high fluorescence intensity can be achieved in staining with the composition of the present invention is not clear, there is a possibility that the length and/or the 3D conformation of —X—Z in Formula (I) is involved.

The composition of the present invention can also stain a gel that has been subjected to Native-PAGE. This is one of the outstanding advantages of the present invention in terms of the limitation of not being able to use the Oriole method, which can carry out one-shot staining.

Another aspect of the present invention relates to a method for analysis of a protein, the method including a step of separating a protein on a support by electrophoresis and a step of carrying out staining and fixation of the protein by immersing the support in an aqueous solution containing the composition of the present invention, but not including a step of destaining the support after immersion.

A typical mode for carrying out the method of the present invention involves, for example, subjecting a sample containing a protein to separation by means of electrophoresis, and then immersing an electrophoresis support such as a gel in an aqueous solution containing a fluorescent compound represented by Formula (I), thus reacting the protein on the support with the fluorescent compound represented by Formula (I).

The composition of the present invention is an aqueous solution (hereinafter, also simply called a ‘staining liquid’); it is typically a buffer, and it is adjusted so as to preferably have a pH of 1 to 4, and more preferably a pH of 2 to 3. The aqueous solution may contain an alcohol. The alcohol is not limited as long as it has high solubility toward water or a buffer, but is preferably an alcohol having 1 to 4 carbon atoms, and more preferably an alcohol having 1 to 3 carbon atoms. The buffer may be any buffer that is usually used in this technical field, such as a phosphoric acid buffer, a citric acid buffer, a glycine buffer, a tris-glycine buffer, a tris buffer, or a MOPS buffer.

The time of immersion in the composition of the present invention is for example 10 to 120 min., preferably no longer than 90 min., and more preferably 30 to 60 min.

In one embodiment of the method of the present invention, instead of immersing an electrophoresis support in the aqueous solution of a fluorescent compound after electrophoresis, the method of the present invention may also be carried out by adding the compound of the present invention to a buffer for electrophoresis, thus carrying out interaction between a protein and the compound of the present invention while electrophoresis is carried out at the same time. This is very advantageous also in terms of giving more accurate analytical results.

In the present invention, the ‘support’ or ‘electrophoresis support’ is not particularly limited as long as it is a support that is usually used in electrophoresis, and examples thereof include a membrane (e.g. a cellulose acetate membrane, a nitrocellulose membrane, a polyvinylidene difluoride (PVDF) membrane, etc.) and a gel (a polyacrylamide gel, an agarose gel, etc.). The support is preferably a gel, and in particular a polyacrylamide gel or an SDS-polyacrylamide gel.

In another embodiment of the method of the present invention, the method of the present invention may also be carried out by contacting a protein in a sample with the compound of the present invention and then carrying out electrophoresis.

In yet another embodiment of the method of the present invention, the method of the present invention may use a method for analysis of a protein that does not employ electrophoresis, such as immunoprecipitation or dot blotting.

Yet another aspect of the present invention relates to a compound represented by Formula I or a salt thereof

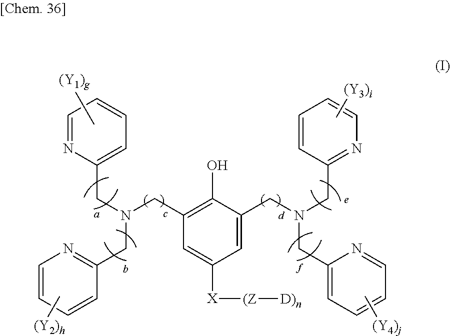

wherein in the Formula,
—X—(Z-D)ₙ is selected from the group consisting of those below

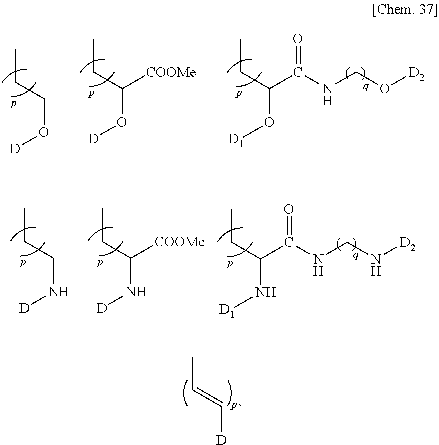

D is selected from the group consisting of

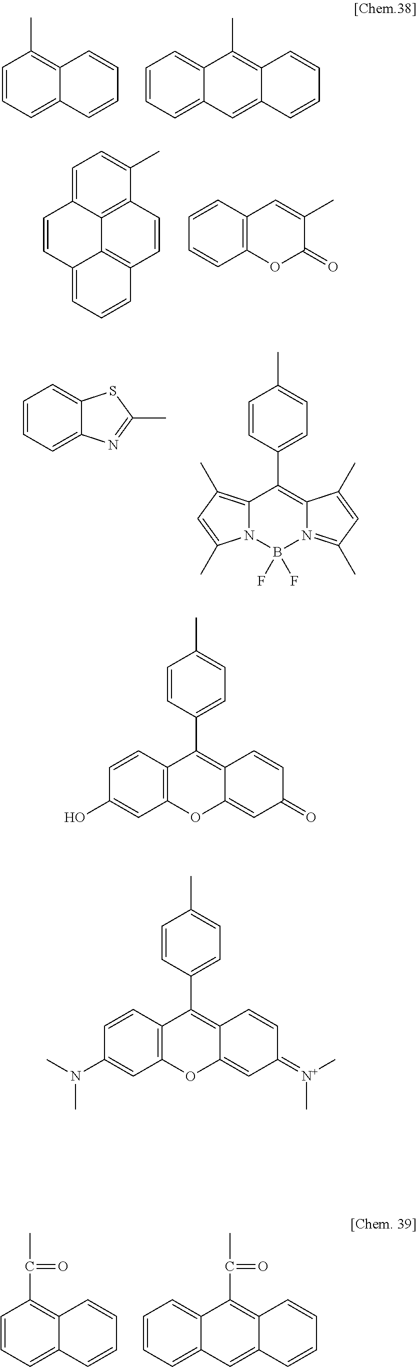

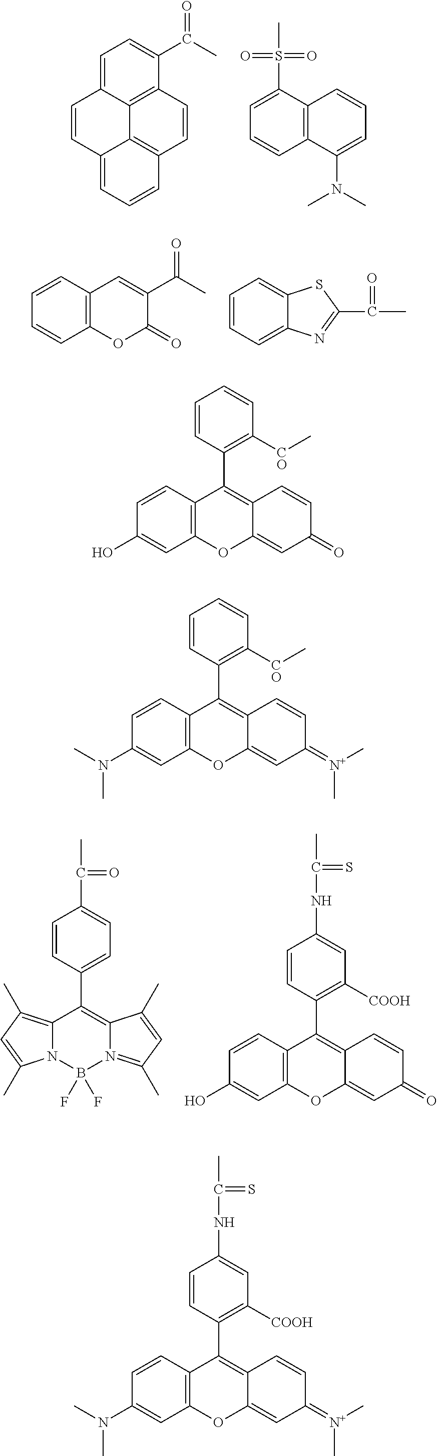

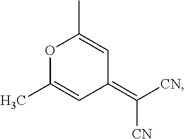

D₁ and D₂ mutually independently denote the same as D, and
p and q are mutually independently a number of 1 to 10.

The compound of the present invention preferably has a structure represented by Formulae Ia to Ic.

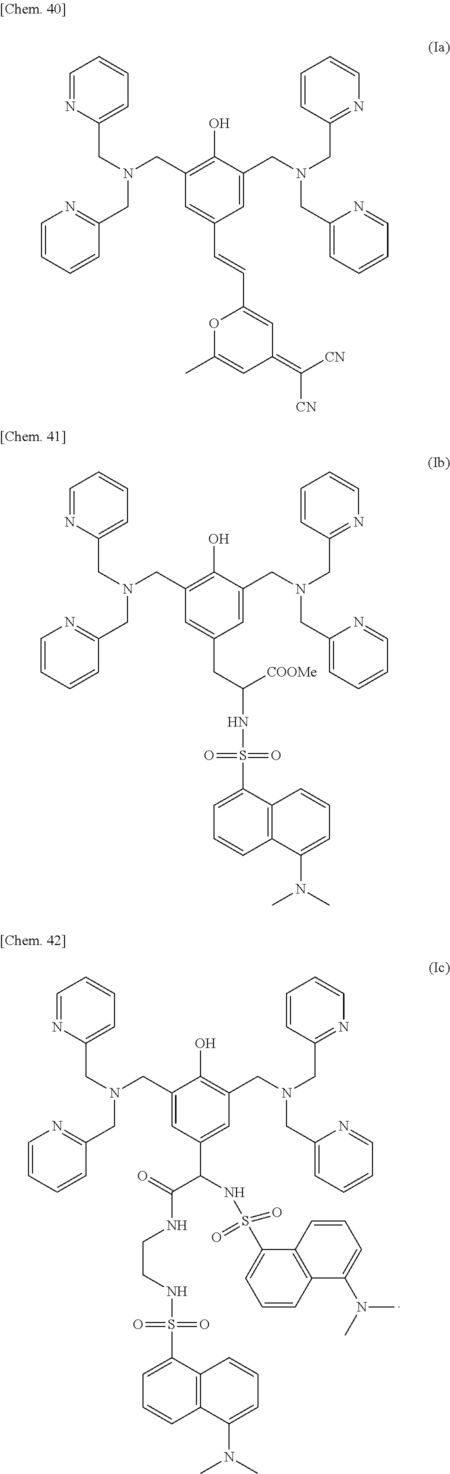 [SMILES: CC1=CC(=C(C#N)C#N)C=C(/C=C/c2cc(CN(Cc3ccccn3)Cc3ccccn3)c(O)c(CN(Cc3ccccn3)Cc3ccccn3)c2)O1 ; COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12 ; CN(C)c1cccc2c(S(=O)(=O)NCCNC(=O)C(NS(=O)(=O)c3cccc4c(N(C)C)cccc34)c3cc(CN(Cc4ccccn4)Cc4ccccn4)c(O)c(CN(Cc4ccccn4)Cc4ccccn4)c3)cccc12]

### EXAMPLES

The present invention is explained below by reference to Examples, but the present invention should not be construed as being limited thereto.

### Example 1

### Method for the synthesis of Fluorescent Substance 1 (Ia)

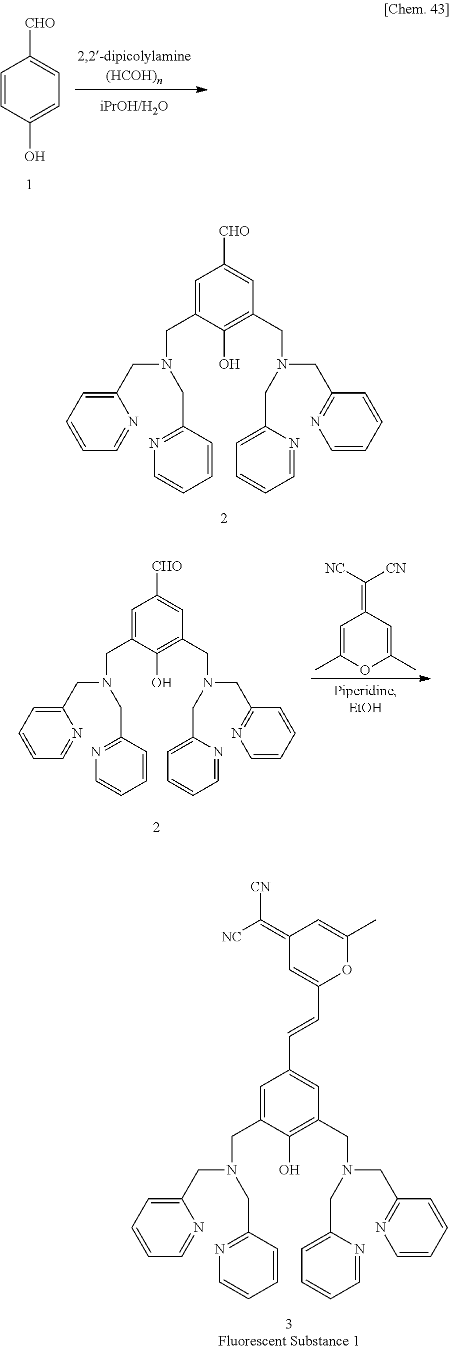

A 100 mL 3-necked flask was charged with 5.0 g (25.3 mmol) of 2,2′-dipicolylamine, 1.2 g (40.4 mmol) of paraformaldehyde, and 72 mL of water/i-PrOH (5:3 v/v), and the pH was adjusted to 8 by adding 1 N HCl. After heating at 80° C. for 30 min., 3.0 g (10.1 mmol) of 4-hydroxybenzaldehyde was added thereto, and the mixture was refluxed for 12 hours. The solvent was removed by distillation under reduced pressure, and the residue was then dissolved in ethyl acetate and washed with a saturated sodium bicarbonate aqueous solution. It was dried with Na₂SO₄, the solvent was then removed by distillation under reduced pressure, and the residue was purified by column chromatography (Al₂O₃, CH₂Cl₂:MeOH=300:10 v/v), thus giving a synthetic intermediate.

Furthermore, a 100 mL pear-shaped flask was charged with 0.5 g (0.9 mmol) of 3,5-bis((bis(pyridin-2-ylmethyl)amino)methyl)-4-hydroxylbenzaldehyde, 0.15 g (0.9 mmol) of 4-(dicyanomethylene)-2,6-dimethyl-4H-pyran, 0.07 g (0.9 mmol) of piperidine, and 50 mL EtOH, and the mixture was refluxed for 12 hours. The solvent was removed by distillation under reduced pressure, and the residue was then dissolved in ethyl acetate and washed with water. It was dried with Na₂SO₄, the solvent was then removed by distillation under reduced pressure, and the residue was purified by column chromatography (Al₂O₃, CH₂Cl₂:MeOH=200:10 v/v), thus giving Fluorescent Substance 1 (Ia).

### [Table 1]

¹H-NMR (CDCl₃, 400 MHz, r.t., TMS, δ/ppm) 2.24 (s, 3H), 3.66 (s, 4H), 3.94 (s, 8H), 5.27 (s, 2H), 6.79˜6.84 (m, 2H), 6.89 (s, 2H), 7.33 (d, 8H), 7.76 (t, 4H), 8.48 (d, 4H), 9.86 (s, 1H)

### Example 2

### Method for the Synthesis of Fluorescent Substance 2 (Ib)

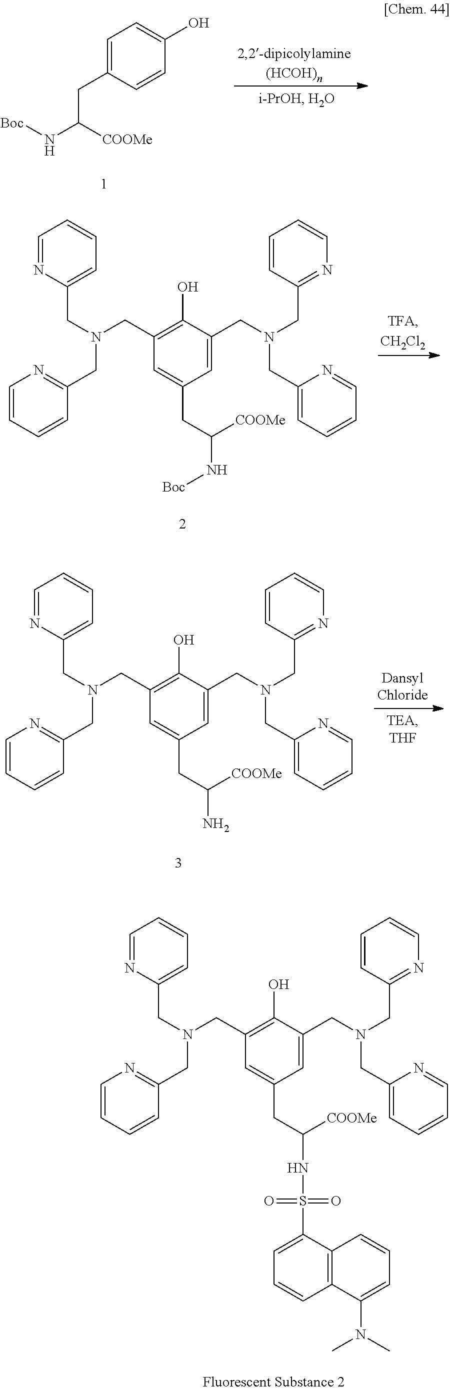 [SMILES: COC(=O)C(Cc1ccc(O)cc1)NC(=O)OC(C)(C)C ; COC(=O)C(Cc1cc(CN(Cc2ccccn2)Cc2ccccn2)c(O)c(CN(Cc2ccccn2)Cc2ccccn2)c1)NC(=O)OC(C)(C)C ; COC(=O)C(N)Cc1cc(CN(Cc2ccccn2)Cc2ccccn2)c(O)c(CN(Cc2ccccn2)Cc2ccccn2)c1 ; COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12]

A 300 mL 3-necked flask was charged with 5.03 g (25.3 mmol) of 2,2′-dipicolylamine, 1.21 g (40.4 mmol) of paraformaldehyde, 62.5 mL of water, 37.5 mL of i-PrOH, and 2.0 mL of 2 N HCl, and it was heated at 80° C. for 30 min. 3.0 g (10.1 mmol) of Boc-L-tyrosine-OMe was added thereto, and the mixture was refluxed for 24 hours. The i-PrOH was removed by distillation under reduced pressure, the residue was then cooled to 0° C., and an oil-like substance that was deposited was collected by separation. It was dissolved in ethyl acetate, then washed in turn with a saturated sodium bicarbonate aqueous solution and saturated brine, and dried with anhydrous sodium sulfate. The solvent was removed by distillation under reduced pressure, and the residue was then purified by column chromatography (Al₂O₃, chloroform:methanol=10:1 v/v), thus giving a brown oil-like compound.

Furthermore, a 50 mL 3-necked flask was charged with 3.6 g (5.1 mmol) of Compound 2 and 25 mL of dichloromethane and placed in an ice bath. 15 mL of trifluoroacetic acid was added over 15 min., the temperature was returned to room temperature, and stirring was carried out for 2 hours. The solvent was removed by distillation under reduced pressure, water was then added thereto, the mixture was made basic using aqueous ammonia, and extraction with dichloromethane was then carried out. The extract was dried with anhydrous sodium sulfate, and the solvent was removed by distillation under reduced pressure, thus giving a synthetic intermediate. Furthermore, a 100 mL 3-necked flask was charged with 0.24 g (0.38 mmol) of Compound 3, 0.04 g (0.45 mmol) of TEA, and 20 mL of THF and placed in an ice bath under a flow of argon. 0.12 g (0.45 mmol) of dansyl chloride was dissolved in 10 mL of THF, and it was then added using a dropping funnel over 30 min. After the mixture was stirred in an ice bath for 30 min., the temperature was returned to room temperature, and stirring was carried out for 12 hours. The solvent was removed by distillation under reduced pressure, and the residue was then dissolved in chloroform and washed with water. It was dried with anhydrous sodium sulfate, the solvent was then removed by distillation under reduced pressure, and the residue was purified by column chromatography (Al₂O₃, CHCl₃:MeOH=10:0.5 v/v), thus giving Fluorescent Substance 2.

### [Table 2]

¹H-NMR (CDCl₃, 400 MHz, r.t., TMS, δ/ppm) 2.79 (6H, s), 3.01 (2H, d), 3.62 (3H, s), 3.78 (4H, s), 3.85 (8H, s), 4.21-4.25 (1H, m), 6.92 (2H, s), 7.05 (1H, d), 7.15 (4H, d), 7.30˜7.40 (2H, m), 7.49 (4H, t) 7.61 (4H, t), 8.24˜8.27 (2H, m), 8.41 (1H, d), 8.56 (4H, d), 11.00 (1H, br s)

### Example 3

### Method for the Synthesis of Fluorescent Substance 3 (Ic)

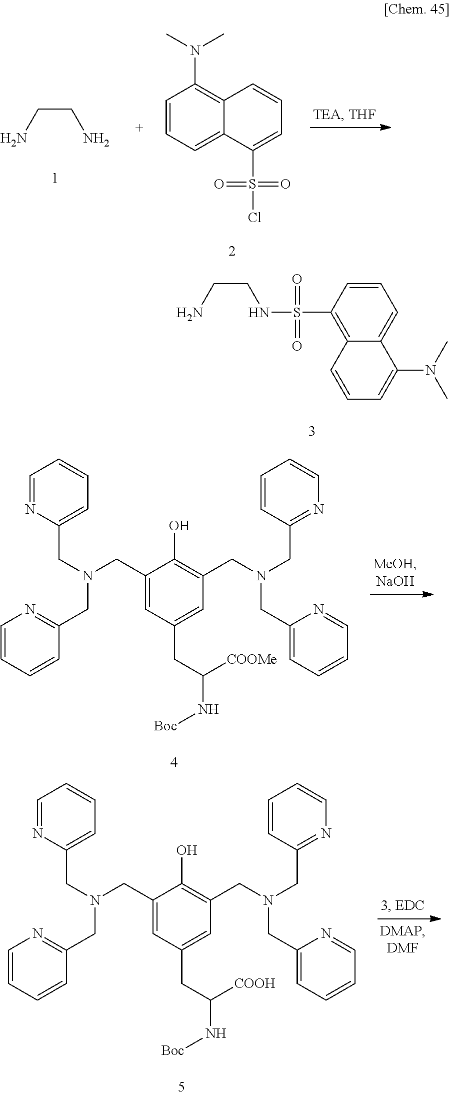

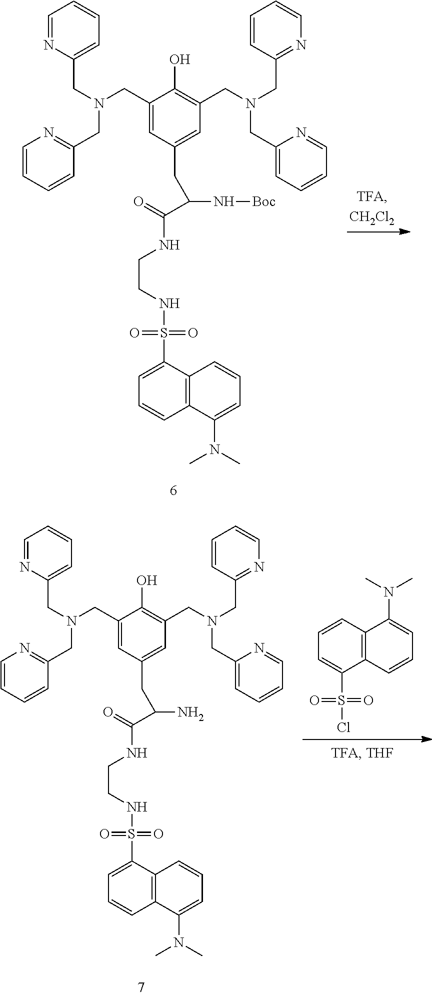

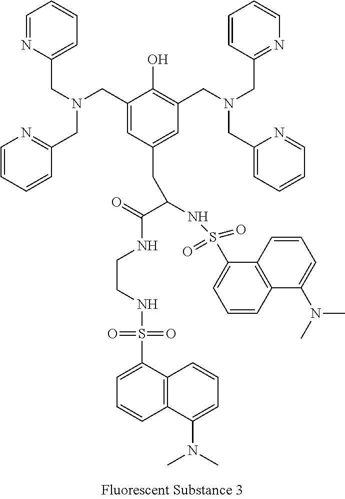 [SMILES: CN(C)c1cccc2c(S(=O)(=O)NCCNC(=O)C(Cc3cc(CN(Cc4ccccn4)Cc4ccccn4)c(O)c(CN(Cc4ccccn4)Cc4ccccn4)c3)NS(=O)(=O)c3cccc4c(N(C)C)cccc34)cccc12]

A 500 mL 3-necked flask was charged with 1.95 g (31.5 mmol) of Compound 1, 3.18 g (31.5 mmol) of triethylamine, and 150 mL of THF and placed in an ice bath. 5.43 g (20.1 mmol) of Compound 2 was dissolved in 50 mL of THF and added thereto using a dropping funnel over 1 hour. The mixture was stirred in an ice bath for 30 min., the temperature was then returned to room temperature, and stirring was carried out for 12 hours. The reaction was stopped by the addition of water, and the solvent was then removed by distillation under reduced pressure. The residue was dissolved in ethyl acetate, then washed with saturated brine, and dried with anhydrous sodium sulfate. The solvent was removed by distillation under reduced pressure, and the residue was then purified by column chromatography (SiO₂, CHCl₃:MeOH=10:1 v/v→acetone:triethylamine=200:5 v/v), thus giving a synthetic intermediate.

Furthermore, a 300 mL 3-necked flask was charged with 6.0 g (8.88 mmol) of Compound 4, 18 mL of a 1 M NaOH aqueous solution, and 100 mL of methanol, and the mixture was stirred at room temperature for 15 hours. The pH was adjusted to 7 to 8 using 1 N HCl, and the solvent was then removed by distillation under reduced pressure. The residue was diluted with water and then washed with diethyl ether, and the pH was adjusted to 4. Extraction with dichloromethane was carried out, and the organic layer was then dried with anhydrous sodium sulfate. The solvent was removed by distillation under reduced pressure, and the residue was then washed in turn with hexane and ether, thus giving a synthetic intermediate.

Furthermore, a 50 mL 3-necked flask was charged with 0.13 g (0.45 mmol) of Compound 3, 0.34 g (0.50 mmol) of Compound 5, 0.24 g (2.0 mmol) of DMAP, and 15 mL of DMF and placed in an ice bath. 0.14 g (0.75 mmol) of EDC was added thereto, stirring was carried out in an ice bath for 30 min., the temperature was then returned to room temperature, and stirring was carried out for 24 hours. The solvent was removed by distillation under reduced pressure, and the residue was then dissolved in chloroform and washed with water. The solvent was removed by distillation under reduced pressure, and the residue was then purified by column chromatography (Al₂O₃, chloroform:methanol=10:1 v/v), thus giving a synthetic intermediate.

Furthermore, a 50 mL 3-necked flask was charged with 0.5 g (0.51 mmol) of Compound 6 and 10 mL of dichloromethane and placed in an ice bath. 1.5 mL of trifluoroacetic acid was added thereto over 15 min., the temperature was returned to room temperature, and stirring was carried out for 2 hours. The solvent was removed by distillation under reduced pressure, water was then added, the mixture was made basic using aqueous ammonia, and extraction with dichloromethane was then carried out. The extract was dried with anhydrous sodium sulfate, and the solvent was then removed by distillation under reduced pressure, thus giving the target compound.

Furthermore, a 100 mL 3-necked flask was charged with 0.45 g (0.51 mmol) of Compound 7, 0.06 g (0.61 mmol) of TEA, and 20 mL of THF and placed in an ice bath. 0.16 g (0.61 mmol) of dansyl chloride was dissolved in 10 mL of THF and then added thereto using a dropping funnel over 10 min. The mixture was stirred in an ice bath for 30 min., the temperature was then returned to room temperature, and stirring was carried out for 12 hours. The reaction was stopped by the addition of water, and the solvent was then removed by distillation under reduced pressure. The residue was dissolved in chloroform and washed with water. It was dried with anhydrous sodium sulfate, the solvent was then removed by distillation under reduced pressure, and the residue was purified by column chromatography (Al₂O₃, CHCl₃:MeOH=100:1 v/v), thus giving Fluorescent Substance 3.

### [Table 3]

¹H-NMR (CDCl₃, 400 MHz, r.t., TMS, δ/ppm) 2.87 (6H, s), 2.90 (6H, s), 3.01 (2H, d), 3.06˜3.08 (2H, m), 3.17˜3.22 (2H, m), 3.79 (4H, s), 3.85 (8H, s), 4.51-4.53 (1H, m) 5.22 (1H, d), 5.38 (1H, t), 5.65 (1H, t), 7.03 (2H, s) 7.15˜7.19 (7H, m) 7.46˜7.58 (7H m) 7.61 (4H, t), 7.63 (1H, d), 8.26˜8.32 (4H, m), 8.54˜8.59 (5H, m), 11.09 (1H, br s)

### Example 4

### Measurement of Wavelength of Absorption Maximum and Wavelength of Fluorescence Maximum

Fluorescent Substance 1 was dissolved in 50 mM MES-NaOH with a pH of 5.9 so as to give concentrations of 50 μg/mL and Fluorescent Substance 2 was dissolved in 50 mM HEPES-NaOH with a pH of 7.2 so as to give concentrations of 100 μg/mL, and absorption spectra were measured. As measurement equipment, a U-3210 (Hitachi High-Technologies Corporation) was used. Furthermore, Fluorescent Substance 1 was dissolved in 50 mM MES-NaOH with a pH of 5.9 so as to give a concentrations of 50 μg/mL and Fluorescent Substance 2 was dissolved in 50 mM HEPES-NaOH with a pH of 7.2 so as to give a concentrations of 5 μg/mL, and fluorescence spectra were measured. As measurement equipment, an RF-1500 (Shimadzu Corporation) was used. The results are given in Table 4. It was confirmed that Fluorescent Substance 1 had optical characteristics suitable for a visible light gel imager and Fluorescent Substance 2 had optical characteristics suitable for a UV transilluminator.

TABLE 4
Optical characteristics of Fluorescent Substances 1 and 2

| | Wavelength of | | Wavelength of |
|---|---|---|---|
| | absorption maximum | | fluorescence maximum |
| Fluorescent | 260, 324, 434 | nm | 566 nm (excitation |
| Substance 1 | | | wavelength: 418 nm) |
| Fluorescent | 304 | nm | 516 nm (excitation |
| Substance 2 | | | wavelength: 304 nm) |

### Example 5

### Staining of Electrophoresis Gel (Fluorescent Substance 1)

### (1) Preparation of Sample

A commercial molecular weight marker (Low Molecular Weight Calibration Kit for SDS Electrophoresis; GE Healthcare) was reconstituted in accordance with a method described in the package insert, subjected to a modification treatment, and then cryopreserved at −80° C. until use. The thawed molecular weight marker was mixed with a sample diluent (a mixed liquid of purified water 3.8 mL, 0.5 M tris-hydrochloric acid buffer (pH 6.8) 1.0 mL, glycerol 0.8 mL, 100 g/L sodium dodecyl sulfate 1.6 mL, and 5 g/L bromophenol blue 0.8 mL), and two-fold serial dilutions were prepared up to a tenth tube. This marker contained Phosphorylase B (97 kDa), Albumin (66 kDa), Ovalbumin (45 kDa), and Carbonic anhydrase (30 kDa).

(2) Separation of Protein by Means of Electrophoresis Conditions for Electrophoresis were as Follows:
Electrophoresis equipment: Rapidas Mini-Slab electrophoresis tank AE6450 (Atto Corporation)

### Polyacrylamide gel: Multi Gel II Mini 10/20 (13W) (Cosmo Bio Co., Ltd.)

Electrophoresis buffer: 25 mM tris(hydroxymethyl)aminomethane, 192 mM glycine, 3.5 mM sodium dodecyl sulfate (pH 8.3)

Each sample of the serial dilutions prepared in (1) above was applied to a gel at 5 μL/well, and electrophoresis was carried out with a constant current of 30 mA for 45 min. The mass of protein applied to the gel was 0.7 to 355 ng/well for Phosphorylase B, 0.8 to 415 ng/well for Albumin, 1.4 to 735 ng/well for Ovalbumin, and 0.8 to 415 ng/well for Carbonic anhydrase.

### (3) Detection of Protein

The gel after electrophoresis was immediately immersed in a staining liquid (a buffer (25 mM phosphoric acid buffer (pH 2.5))/purified water/methanol=9/9/2 (v/v)) in which 16 μg/mL of Compound 1 had been dissolved) for 30 min. An image was taken using a gel imager while illuminating with visible light.

Gel imager: Light Capture II (Atto Corporation)
Light source: green LED (525 nm)
Cut-off filter: R-60

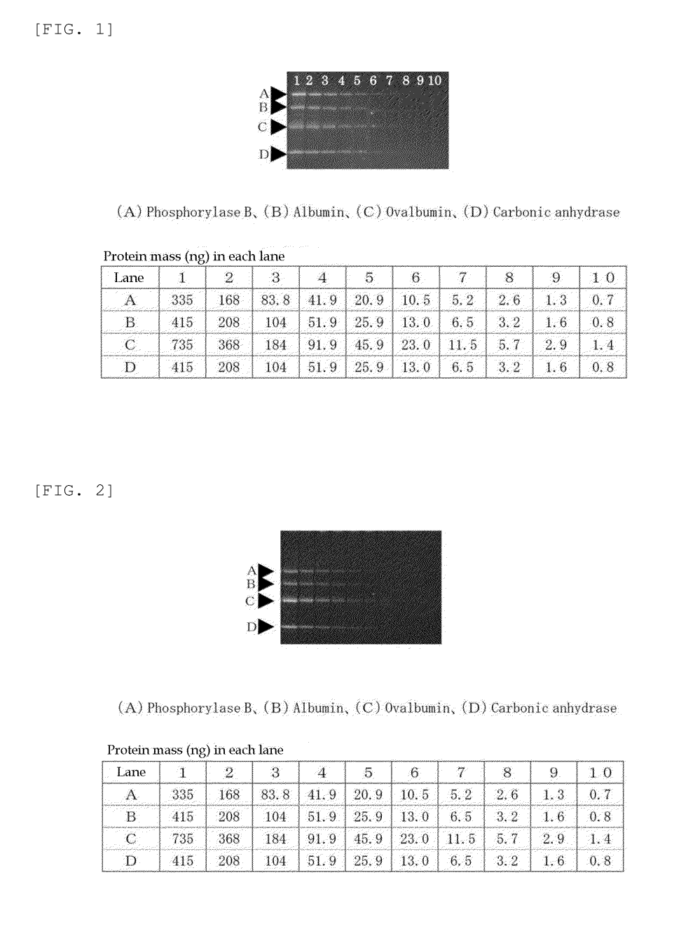

The results are given in Table 5 and FIG. 1. The time taken for staining the protein was only 30 min., and the operation was only a single step of immersing the gel in the staining liquid, but detection was carried out with the same level of sensitivity as that of silver staining.

TABLE 5
Results of detection of protein by Fluorescent Substance 1
(Staining liquid: phosphoric acid buffer/purified
water/methanol)

| Protein | Detection sensitivity (ng) |
|---|---|
| Phosphorylase B | 1.3 |
| Albumin | 3.2 |
| Ovalbumin | 5.7 |
| Carbonic anhydrase | 3.2 |

### Example 6

### Staining of Electrophoresis Gel (Fluorescent Substance 2)

### (1) Preparation of Sample

A sample was prepared in accordance with the analytical conditions described in Example 5.

### (2) Separation of Protein by Means of Electrophoresis

Separation of protein by means of electrophoresis was carried out in accordance with the analytical conditions described in Example 5.

### (3) Detection of Protein

The gel after electrophoresis was immediately immersed in a staining liquid (a buffer (25 mM glycine buffer (pH 2.5))/purified water/ethanol=9/9/2 (v/v)) in which 16 μg/mL of Fluorescent Substance 2 had been dissolved) for 30 min. An image was taken using a gel imager while illuminating with UV.

Gel imager: Printgraph (Atto Corporation)
Light source: UV (365 nm)
Cut-off filter: SCF515

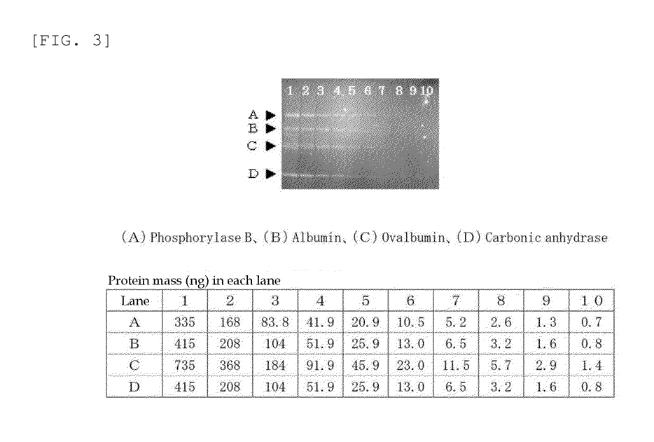

The results are given in Table 6 and FIG. 2. The time taken for staining the protein was only 30 min., and the operation was only a single step of immersing the gel in the staining liquid, but detection was carried out with the same level of sensitivity as that of silver staining.

TABLE 6
Results of detection of protein by Fluorescent Substance 2
(Staining liquid: glycine buffer/purified water/ethanol)

| Protein | Detection sensitivity (ng) |
|---|---|
| Phosphorylase B | 1.3 |
| Albumin | 1.6 |
| Ovalbumin | 2.9 |
| Carbonic anhydrase | 1.6 |

### Example 7

### Staining of Electrophoresis Gel (Fluorescent Substance 3)

A sample was prepared in accordance with the analytical conditions described in Example 5, and separation of a protein by means of electrophoresis was carried out. The gel after electrophoresis was immediately immersed in a staining liquid (25 mM glycine buffer (pH 2.5)/purified water/ethanol=9/9/2 (v/v)) in which Fluorescent Substance 3 (16 μg/mL) had been dissolved for 30 min. An image was taken using a gel imager while illuminating with UV.

### Gel Imager: Printgraph (Atto Corporation)

### Light Source: UV (365 nm)

### Cut-off Filter: SCF515

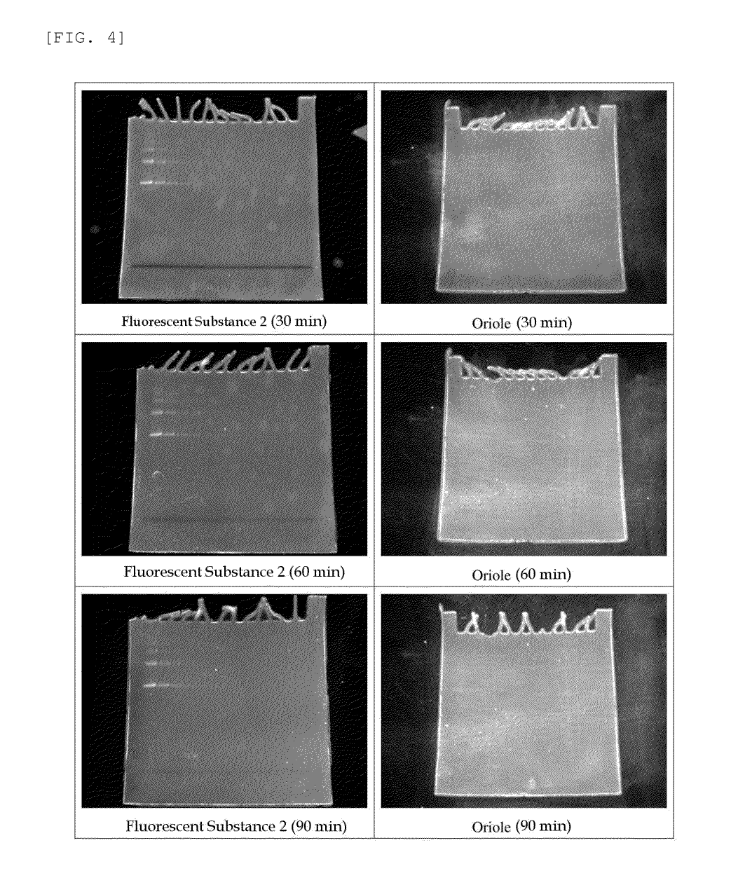

The results are given in Table 7 and FIG. 3. The time taken for staining the protein was only 30 min., and the operation was only a single step of immersing the gel in the staining liquid, but detection was carried out with the same level of sensitivity as that of silver staining. However, the background was high.

TABLE 7
Results of detection of protein by Fluorescent Substance 3
(Staining liquid: glycine buffer/purified water/ethanol)

| Protein | Detection sensitivity (ng) |
|---|---|
| Phosphorylase B | 2.6 |
| Albumin | 3.2 |
| Ovalbumin | 5.7 |
| Carbonic anhydrase | 1.6 |

### Example 8

### Staining of Native-PAGE Gel (Fluorescent Substance 2)

A Native-PAGE gel containing Bovine Serum Albumin (hereinafter, BSA) as a sample was subjected to staining with a one-shot protein staining reagent (Fluorescent Substance 2), and an image was taken by a gel imager. As a comparative control, a gel was stained in the same manner using Oriole.

### (1) Preparation of Sample

BSA (Proliant) was dissolved in a 1× sample loading buffer for Native-PAGE {62.5 mM Tris-HCl pH 6.8, 20% (v/v) glycerol, 0.01% (w/v) bromophenol blue} at a concentration of 1 mg/mL, and this was defined as a first tube. For a second tube and thereafter, 2-fold serial dilutions of the 1× sample loading buffer were prepared up to a 13^{th} tube.

### (2) Native-PAGE

5 μL of the samples of the 1^{st} to 13^{th} tubes were applied to 1^{st} to 13^{th} lanes respectively of a Multi Gel II Mini 10/20 (13 W) (Cosmo Bio Co., Ltd.), and the protein was separated by means of Native-PAGE (15 mA per sheet of gel, 90 min.).

### (3) Detection of Protein

The gel was immediately immersed in the one-shot protein staining reagent or Oriole, and images of the gel were taken every 30 min.

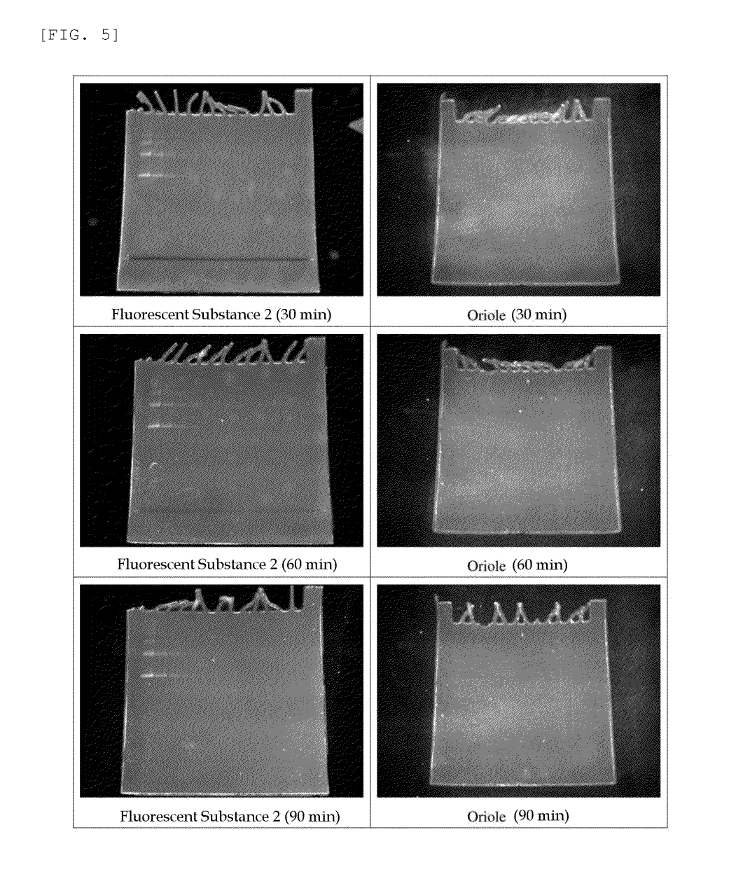

As shown in FIGS. 4 and 5, in the case of the one-shot protein staining reagent, a ladder-shaped band was detected. It is thought that the reason why a single band was not obtained is because of formation of a multimer of BSA or a higher-order structure. On the other hand, in the case of Oriole, no band was detected.

### INDUSTRIAL APPLICABILITY

The method of the present invention for analysis of a protein enables various types of proteins to be analyzed equally, and in accordance with use of the method of the present invention, it is useful for rapid, high-sensitivity, and simple analysis of proteins in the fields of for example biochemistry, medicine, food, analytical chemistry, etc.

1. A composition for analysis of a protein, comprising one or more types of compound represented by Formula I and/or salt thereof 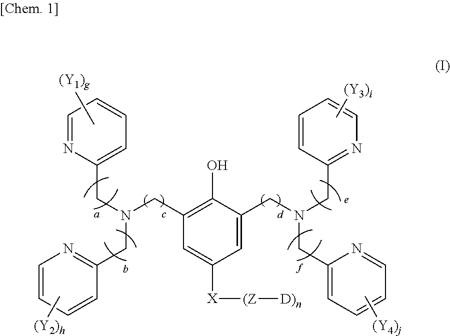
wherein in the Formula a to f are mutually independently 1 to 5,
Y₁ is a straight-chain or branched alkyl group having 1 to 10 C atoms, a carboxyl group, a hydroxy group, an amino group, a thiol group, or a halogen, and when a plurality of Y₁s are present they may be mutually independently identical or different,
Y₂ to Y₄ mutually independently denote the same as Y₁,
g, h, i, and j are mutually independently 0 to 4,
X is a straight-chain hydrocarbon group having 1 to 10 C atoms in which one or more hydrogen atoms are mutually independently and optionally replaced by —COOH, —OH, —NH₂, —R₁, —OR₁, —COR₁, —COOR₁, —CONHR₁, —SH, or a halogen, R₁ being an alkyl group having 1 to 5 C atoms, and at least one hydrogen atom bonded to a terminal carbon atom of the hydrocarbon group being replaced by —Z-D,
n is 1 to 3,
z is a single bond or an alkylene group, when a plurality of Zs are present they are independent from each other, one or more —CH₂— moieties being mutually independently and optionally replaced by —O—, —S—, —NH—, —NH—SO₂—, —SO₂—NH—, —CS—NH—, —NH—CS—, —CO—NH—, —NH—CO—, —CO—, —COO—, —OCO—, —OCO—O—, —S—CO—, —CO—S—, —SO₂—, —CH═CH—, or —C≡C—, and
D is a chromophore, when a plurality of Ds are present they are independent from each other,
when the composition is applied to a support containing a separated protein, staining and fixation of the protein being carried out thereby, and no destaining of the support after application being required.

2. The composition according to claim 1, wherein D is selected from the group consisting of

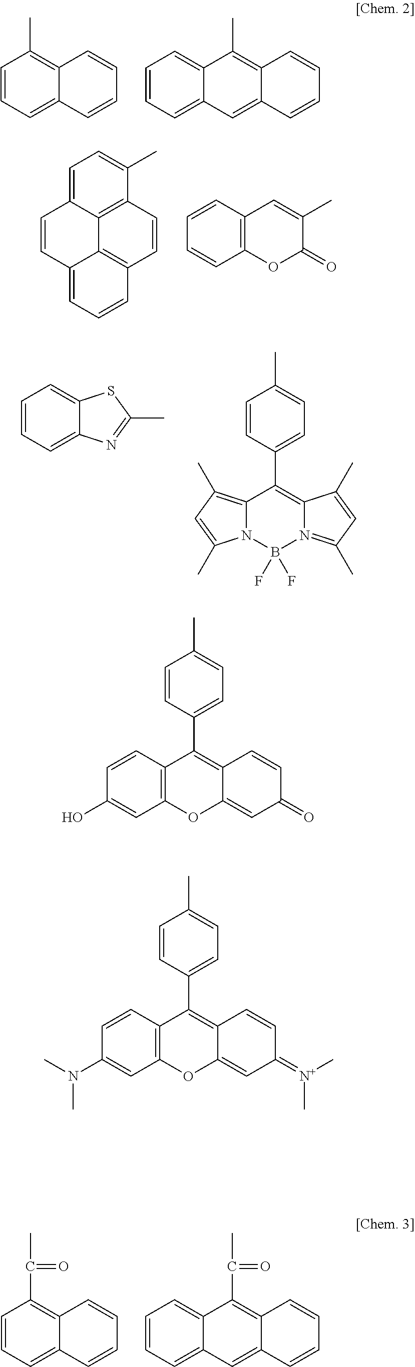

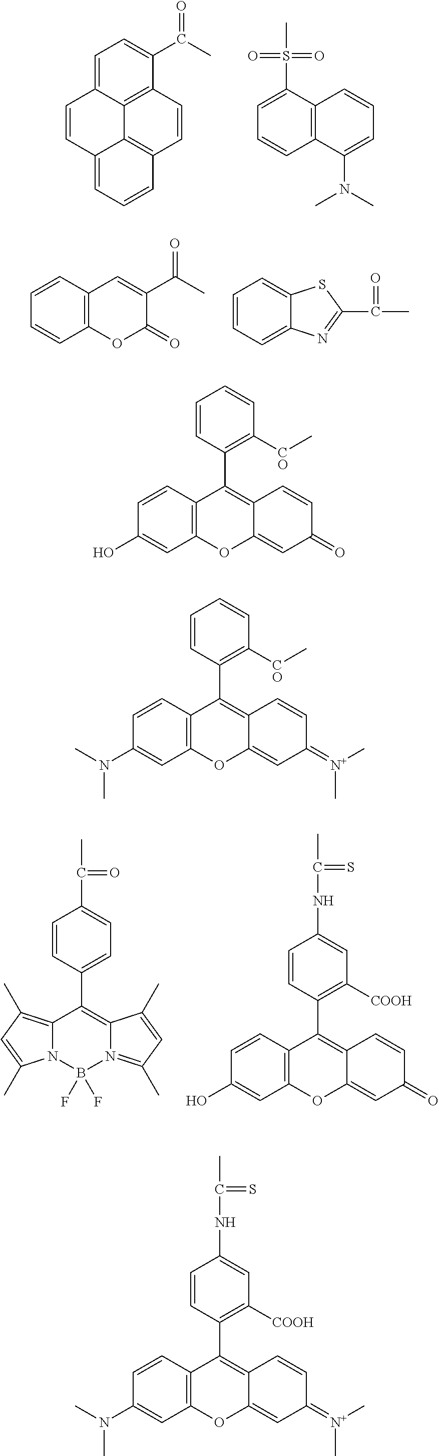

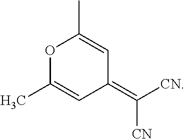

3. The composition according to claim 1, wherein —X—(Z-D)ₙ is selected from the group consisting of those below 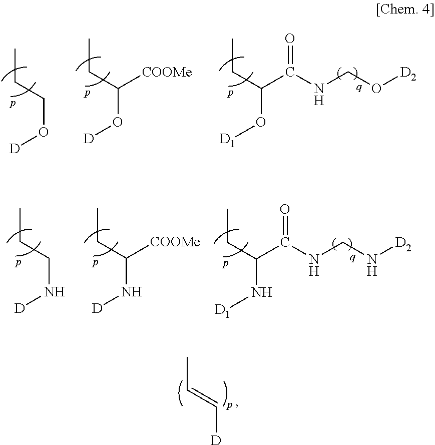
D is selected from the group consisting of 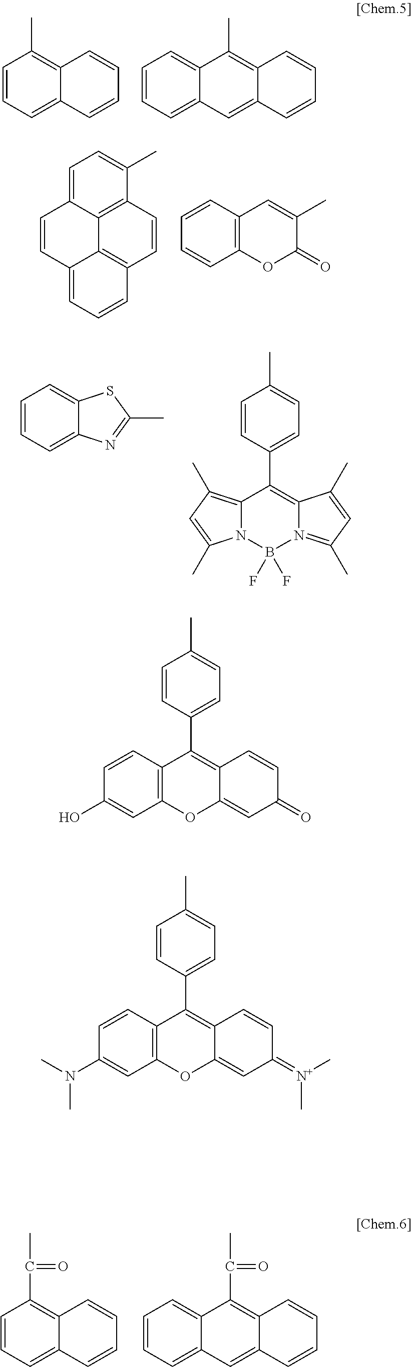 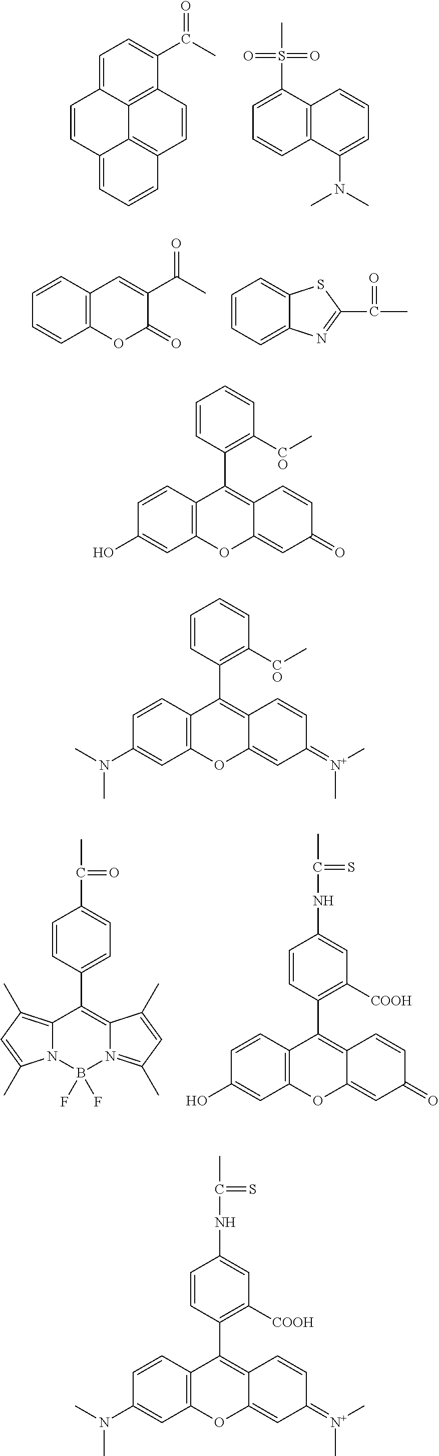 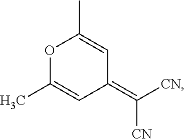
D₁ and D₂ mutually independently denote the same as D, and
p and q are mutually independently a number of 1 to 10.

4. The composition according to claim 1, wherein it comprises at least one type of compound represented by Formulae Ia to Ic and/or salt thereof

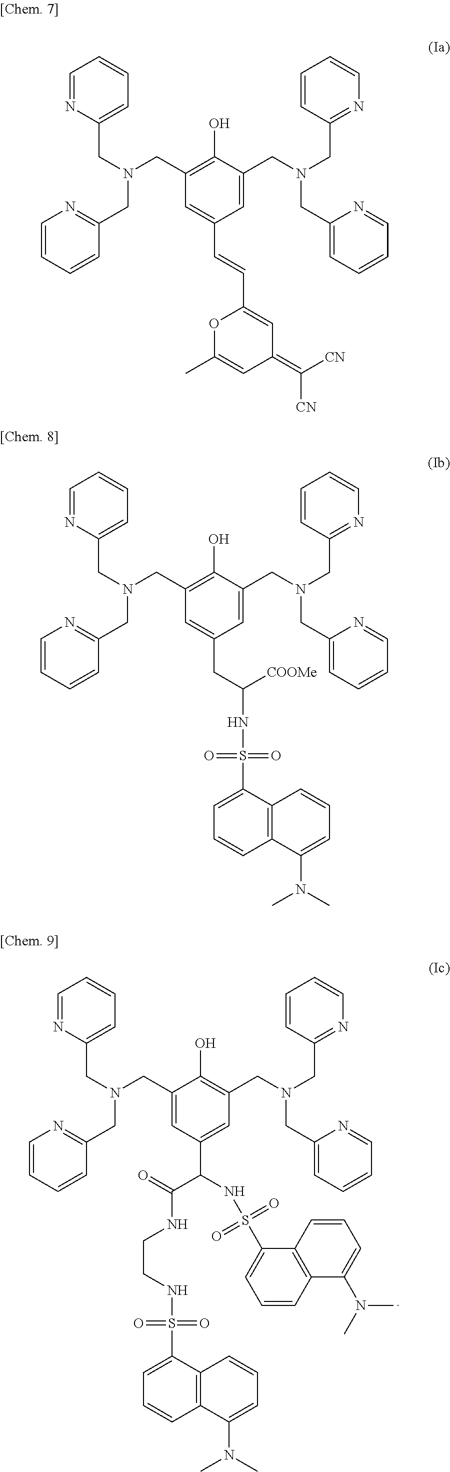 [SMILES: CC1=CC(=C(C#N)C#N)C=C(/C=C/c2cc(CN(Cc3ccccn3)Cc3ccccn3)c(O)c(CN(Cc3ccccn3)Cc3ccccn3)c2)O1 ; COC(=O)C(Cc1cc(CN(Cc2ccccn2)Cc2ccccn2)c(O)c(CN(Cc2ccccn2)Cc2ccccn2)c1)NS(=O)(=O)c1cccc2c(N(C)C)cccc12 ; CN(C)c1cccc2c(S(=O)(=O)NCCNC(=O)C(NS(=O)(=O)c3cccc4c(N(C)C)cccc34)c3cc(CN(Cc4ccccn4)Cc4ccccn4)c(O)c(CN(Cc4ccccn4)Cc4ccccn4)c3)cccc12]

5. A method for analysis of a protein, the method comprising a step of separating a protein on a support by means of electrophoresis and a step of carrying out staining and fixation of the protein by immersing the support in an aqueous solution containing the composition according to claim 1, but not comprising a step of destaining the support after immersion.

6. The method according to claim 5, wherein it further comprises a step of subjecting the support after immersion to fluorescence image analysis by illuminating with visible light or UV.

7. A compound represented by 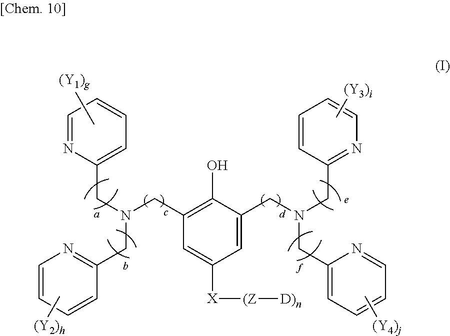
or a salt thereof,
wherein in the Formula
—X—(Z-D)ₙ is selected from the group consisting of those below 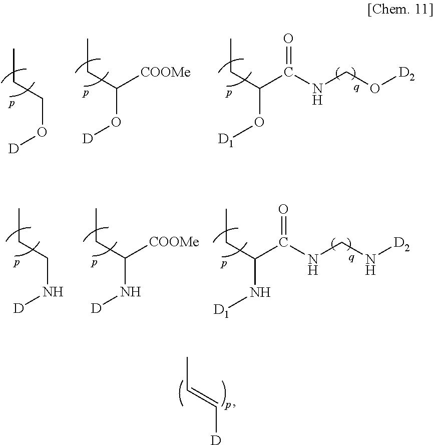
D is selected from the group consisting of 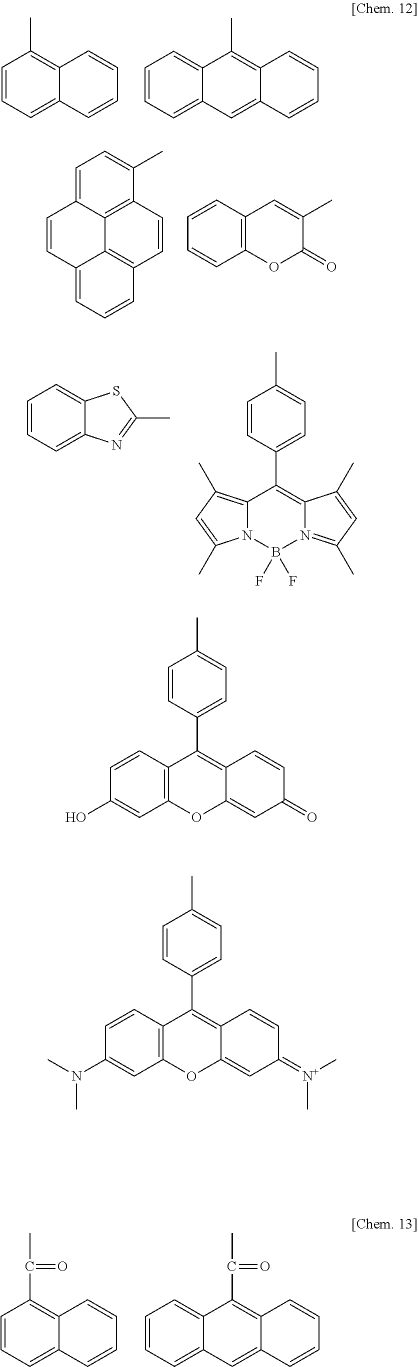 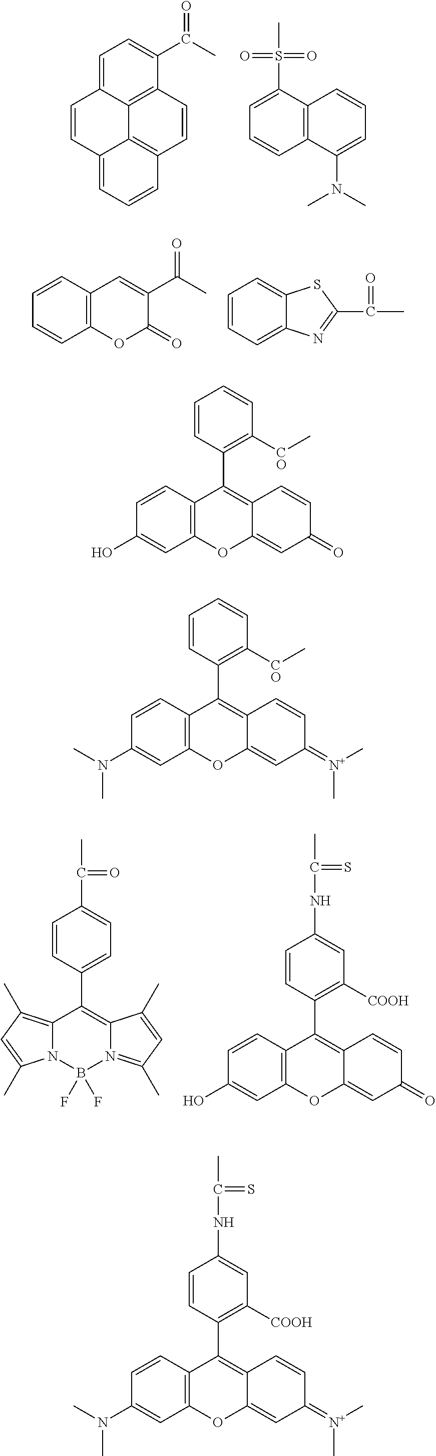 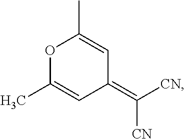
D₁ and D₂ mutually independently denote the same as D, and
p and q mutually independently denote a number of 1 to 10.

8. The compound or salt thereof according to claim 7, wherein the compound is represented by Formula Ia

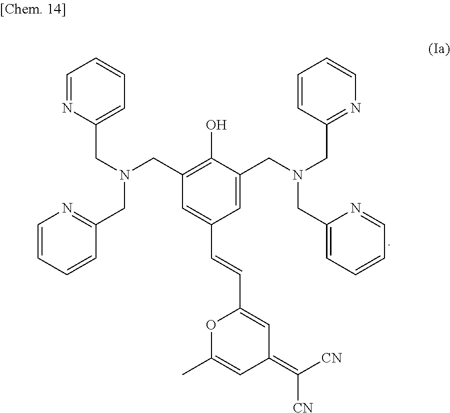 [SMILES: CC1=CC(=C(C#N)C#N)C=C(/C=C/c2cc(CN(Cc3ccccn3)Cc3ccccn3)c(O)c(CN(Cc3ccccn3)Cc3ccccn3)c2)O1]

9. The compound or salt thereof according to claim 7, wherein the compound is represented by Formula Ib

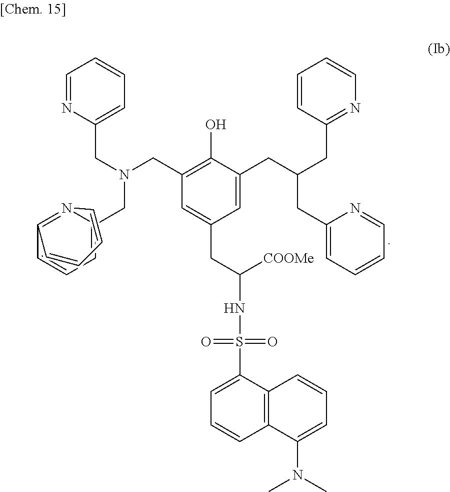

10. The compound or salt thereof according to claim 7, wherein the compound is represented by Formula Ic 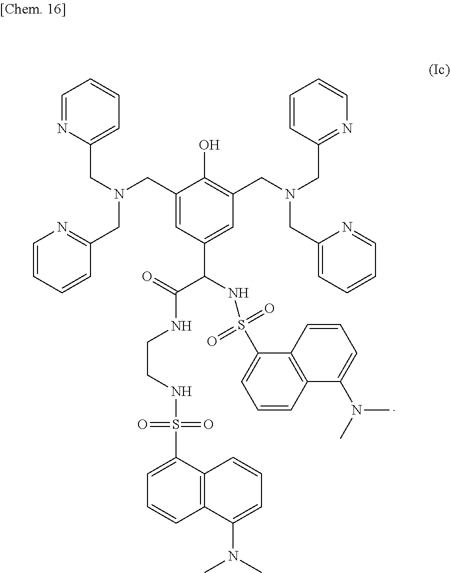 [SMILES: CN(C)c1cccc2c(S(=O)(=O)NCCNC(=O)C(NS(=O)(=O)c3cccc4c(N(C)C)cccc34)c3cc(CN(Cc4ccccn4)Cc4ccccn4)c(O)c(CN(Cc4ccccn4)Cc4ccccn4)c3)cccc12]
